(19) United States
(12) Patent Application Publication
(10) Pub. No.: US 2026/0308590 A1
(43) Pub. Date: Oct. 8, 2026

(54) METHODS OF TREATMENT OF CHRONIC KIDNEY DISEASE

(71) Applicant: ASTRAZENECA AB, Södertälje (SE)

(72) Inventors: Lisa BUVALL, Södertälje (SE); Lars Anders DAHLÉN, Södertälje (SE); Hans Fredrik KARLSSON, Södertälje (SE); Alex Xianghua ZHOU, Södertälje (SE); Hanna WALLENTIN, Södertälje (SE); Karl Henrik EDMAN, Södertälje (SE); Stefan GESCHWINDNER, Södertälje (SE); Mats Fredrik ORMO, Södertälje (SE)

(21) Appl. No.: 19/483,143

(22) PCT Filed: May 10, 2024
(86) PCT No.: PCT/EP2024/062974

§ 371 Date: Nov. 10, 2025

Related U.S. Application Data
(60) Provisional application No. 63/501,462, filed on May 11, 2023.

(51) Int. Cl. C12N 15/113; A61K 31/7115; A61K 31/712; A61P 13/12

(52) U.S. Cl. CPC C12N 15/113; A61K 31/7115; A61K 31/712; A61P 13/12; C12N 2310/11; C12N 2310/3231; C12N 2310/3341; C12N 2310/341

(57) ABSTRACT

The application relates to methods of treating chronic kidney disease in a patient in need thereof involving administering an inhibitor of ARGHEF6 activity such as an antisense oligonucleotide. Use of ARHGEF6 inhibitors in medicine and in the manufacture of medicines are also disclosed.

The present specification relates to a method of treatment or prophylaxis of chronic kidney disease (CKD) comprising administration of an inhibitor of ARHGEF6 activity to a patient in need thereof. The inhibitor of ARHGEF6 activity may work by depleting or degrading ARHGEF6 at a protein level or by binding to ARHGEF6 and in doing so blocking the activation of Rac1 and/or the reduction of active (1-integrin levels mediated by ARHGEF6. The specification also relates to an inhibitor of ARHGEF6 activity for use in the treatment of chronic kidney disease. Methods of identifying a patient for treatment or prophylaxis with an inhibitor of ARHGEF6 activity comprising the step of identifying a biomarker of CKD in a sample obtained from said patient are also provided and may be incorporated into the overall method of treatment or prophylaxis.

Chronic kidney disease (CKD) is a broad term used to describe diseases of the kidney that are characterised by a gradual loss, and ultimately failure, of kidney function. As CKD progresses a kidney's ability to filter waste and excess fluid from the blood diminishes. CKD is classified on a scale ranging from stage 1 to stage 5 based on the level of kidney damage a patient has sustained and the resultant decline in their measured glomerular filtration rate (GFR) (see KDIGO—Kidney International Supplements 2013, 3, 19-62 and Kidney International (2024), 105 (Suppl 4S), S117-S314), with stage 5 CKD, also referred to as end stage renal disease (ESRD) or kidney failure, being the most severe. The GFR can be estimated, with estimated GFR (eGFR) values being derivable from measurement of filtration markers such as serum creatinine or cystatin C. eGFR is a measure of the kidney's ability to clean the blood relative to the expected “normal” value, albeit kidney function varies according to age, sex, and body size, and declines with age (for illustration, the “normal” eGFR of a 40 year old is around 100, while the normal value for a 70 year old is around 75). Various approaches for establishing eGFR are described in the literature and are well known to those skilled in the art (see e.g. L. A. Inker et al, N Engl J Med 2021; 385:1737-1749, DOI: 10.1056/NEJMoa2102953; N Engl J Med 2012; 367:20-29, DOI: 10.1056/NEJMoa1114248 and the KDIGO Guidelines referred to above).

In the early stages of CKD (stages 1 and 2) a patient may display few signs or symptoms of the condition, the latter being relatively mild (e.g. high blood pressure, swelling of the legs and urinary tract infections). Stage 1 and 2 CKD patients have sustained kidney damage, but have apparently normal, or only mildly impaired, kidney function (stage 1 eGFR=90% or higher/stage 2 CKD eGFR=89 to 60). By stage 3 CKD however, patients have mild to moderate (stage 3a, eGFR=45 to 59) or moderate to severe (stage 3b, eGFR=30 to 44) loss of kidney function and experience an increased number of, and severity of, symptoms (e.g. low blood count, malnutrition, bone pain, unusual pain, numbness or tingling, decreased mental sharpness or feeling unwell). Stage 3 CKD patients also have an increased propensity to progress to stage 4 (eGFR=29 to 15) or stage 5 CKD (also known as end stage renal disease (ESRD) or kidney failure, at which stage the eGFR is less than 15). By stage 4 CKD, patients have experienced a severe loss in kidney function and are prone to anaemia, decreased appetite, bone disease or abnormal blood levels of phosphorus, calcium or vitamin D. Additional symptoms associated with stage 5 CKD/ESRD include uraemia, fatigue, shortness of breath, nausea, vomiting, abnormal thyroid levels, swelling in hands/legs/eyes/lower back or lower back pain. Morbidity and mortality rates for stage 4 and stage 5 CKD patients are high.

Guidelines for the definition and classification of CKD can be found in Kidney International Supplements (2013) 3, 19-62 (KDIGO) and, more recently in the 2024 KDIGO guidelines, see Kidney International (2024), 105 (Suppl 4S), S117-S314 collectively referred to as KDIGO/KDIGO guidelines herein. According to the KDIGO guidelines, GFR is accepted as the best overall index of kidney function because a) it is generally reduced after widespread structural kidney damage and b) most other kidney functions decline in parallel with GFR in CKD. A GFR of <60 ml/min/1.73 m² for >3 months was selected as indicative of CKD, this value is less than half of the normal value in young adult men and women (125 ml/min/1.73 m²). A GFR of 60 ml/min/1.73 m² can be detected by routine laboratory testing.

Current estimating equations for GFR (eGFR) based on serum creatinine (sCr), but not sCr alone, are sensitive for detecting measured GFR. A decreased eGFR established using sCr can be confirmed by GFR estimation using an alternative filtration marker (cystatin C) or GFR measurement, as necessary.

Currently, treatment options for patients with severe or late stage renal disease include dialysis and kidney transplantation. These treatments are intrusive, expensive and not universally available since highly developed medical systems are required for their delivery. Consequently, there is a substantial medical need to detect CKD at as early a stage as possible and provide treatments that can slow, arrest or even reverse progression of the disease. Identification of new effective strategies for therapeutic intervention in the treatment of CKD are therefore required, as are biomarkers prognostic of susceptibility to the development, initiation, presence or progression of kidney disease.

On a functional level, the kidney removes waste and excess fluid from the blood through the glomerular filtration barrier into the urinary tract for excretion. The glomerular filtration barrier is a complex structure comprising, among other elements, podocytes attached to the glomerular basement membrane (GBM). In this structure, podocytes are tightly clustered and adopt a special shape such that a slit between adjacent podocytes is formed and maintained. This slit between adjacent podocytes, sometimes referred to as the filtration slit or slit diaphragm, is spanned by various intracellular elements such as nephrin to form a matrix through which controlled diffusion of waste and excess fluid from the blood into the urinary tract can occur. The shape of the podocytes on the GBM is maintained by the podocytes' internal actin cytoskeleton (composed of dynamic actin stress fibres). It follows that the integrity of the podocytes' actin cytoskeleton is required for optimal kidney function. In addition to their shape, the podocytes are anchored on to the GBM through various factors such as dystroglycan, syndecan and α3 and β1-integrins. Consequently, maintenance of the anchoring of podocytes is also a key factor in the integrity of the glomerular filtration barrier and kidney function. Efficient functioning of the glomerular filtration barrier requires both stable anchoring of podocytes to the glomerular filtration barrier and the maintenance of podocyte shape.

Guanine nucleotide exchange factors (GEFs) are proteins or protein domains that activate monomeric GTPases by stimulating the release of guanosine diphosphate (GDP) to allow binding of guanosine triphosphate (GTP). Rho guanine nucleotide exchange factor 6 (ARHGEF6) is a GEF protein encoded in humans by the ARHGEF6 gene (NB. for the avoidance of doubt, ARHGEF6 (in italics) is used to denote the gene herein, while ARHGEF6 is used to denote protein). Amongst other names (e.g. PIXA; COOL2; MRX46; Cool-2; alphaPIX; alpha-PIX) ARHGEF6 is also referred to as Rac/Cdc42 guanine nucleotide exchange factor 6 as it characteristically binds to the GTPases Cdc42 and Rac1. Formation of a stable complex between ARHGEF6 and activated Cdc42 was observed to enhance ARHGEF6's ability to associate with GDP bound Rac1 (see Baird et al, Current Biology 2001, 15, 1-10). The GTPases Cdc42 and Rac1 that bind to ARHGEF6 are members of the Rho subfamily of Ras-related GTP binding proteins and are implicated in a wide variety of cellular responses, including the regulation of the actin cytoskeletal architecture, cell shape and motility, intracellular trafficking, cell cycle progression, and malignant transformation. The catalytic subunit of ARGHEF6 consists of a Dbl-homology domain (DH) and a pleckstrin-homology domain (PH). Studies have suggested that the PH domain stabilises the DH domain in its binding to Rac1, with the catalytic activity of constructs comprising a DH domain and lacking a PH domain proving greatly reduced. A precise mechanistic understanding of the ARGHEF6 mediated conversion of Rac1-GDP to Rac1-GTP however remains to be established and it may be that ARHGEF6 acts upstream of the final conversion step.

ARHGEF6 knockdown in mice and consequent loss of ARHGEF6 impairs hair cell stereocilia development and results in progressive hearing loss (Zhu et al, Mol. Neurosci. 2018, 11:362). To the best of our knowledge, prior to the present specification no association between ARHGEF6 activity and the development and/or progression of chronic kidney disease (CKD)/end stage renal disease (ESRD) has been established. Furthermore, prior to the present specification there has been no suggestion that inhibiting ARHGEF6 activity could represent a therapeutic strategy for the treatment of CKD/ESRD. Accordingly, it is an object of the present specification to provide a new method of treatment or prophylaxis for CKD/ESRD involving inhibiting ARHGEF6 activity, for example in podocytes.

In a first aspect of the specification there is provided a method for treatment or prophylaxis of chronic kidney disease comprising administration of an inhibitor of ARHGEF6 activity to a patient in need thereof. The inventors have identified that upregulation of ARHGEF6 mRNA is detrimental to podocyte morphology and also the attachment of podocytes to the glomerular basement membrane (GBM). The evidence presented herein indicates that the upregulation of ARHGEF6 (i) has an effect on the actin cytoskeleton of podocytes through Rac1 activation and (ii) causes depletion of active (1-integrin levels thus compromising the attachment of podocytes to the GBM. Furthermore, reduction of ARHGEF6 at a protein level has been shown to restore the podocytes normal morphology and restore active (β1-integrins levels. In addition to its effect on podocytes, ARHGEF6 overexpression in primary human glomerular endothelial cells (HGMEC) was found to significantly increase apoptosis suggesting that upregulated ARHGEF6 may partially contribute to endothelial dysfunction in CKD.

Accordingly, in embodiments the methods of treatment or prophylaxis of chronic kidney disease comprise use of agents capable of reducing the amount of ARHGEF6 at a protein level. In addition, in embodiments the methods of treatment or prophylaxis of chronic kidney disease comprise use of agents that reduce the ARHGEF6 mediated activation of Rac1 and the ARHGEF6 mediated depletion of active (β1-integrin levels. In embodiments the patient for treatment is a human patient. In embodiments the inhibitor of ARHGEF6 activity for use in treatment is intended for use in a human patient. In certain embodiments the inhibitor of ARHGEF6 activity for use in the manufacture of a medicament is an inhibitor of ARHGEF6 activity for human use.

In a second aspect there is provided an inhibitor of ARHGEF6 activity for use in the treatment or prophylaxis of chronic kidney disease. In embodiments the inhibitor of ARHGEF6 activity is an agent capable of reducing the amount of ARHGEF6 at the protein level.

In a third aspect there is provided an inhibitor of ARHGEF6 activity for use in the manufacture of a medicament for the treatment of chronic kidney disease.

In a further aspect there is provided a an antisense oligonucleotide that selectively binds to ARHGEF6 mRNA, thereby silencing ARHGEF6 protein expression.

In a further aspect there is provided a proteolysis targeting chimera (PROTAC) or a small molecule degrader, each of which elicits degradation of ARHGEF6 protein.

### FIGURES

So that the specification may be better understood, the following figures are referred to herein:

FIG. 1: Lollipop plot of the positions of the rare missense mutations in ARHGEF6, Rac/Cdc42 guanine nucleotide exchange factor 6 obtained in a study comparing the genomes of ca 500 CKD patients with ca 9,000 controls. The six rare missense mutations in CKD patients (above the line, in pink) are indicated above the plot with 5 out of 6 of these being found in the DH domain. Missense mutations in the control population, indicated below the line (in blue), are in contrast widely distributed across the protein structure. The domains in the figure, working from left to right in order are: (A) Calponin homology (CH) domain; (B) RhoGEF67_u1; (C) SH3_9; (D) RhoGEF domain; (E) PH domain; (F) RhoGEF67_u2; (G) betaPIX coiled coil.

FIG. 2: FIG. 2a) A Western blot using ARHGEF6 antibody shows equal expression and cleavage of the expressed 87 kDa isoform to the shorter 71 kDa isoform, across wild type (wt) and ARHGEF6 mutants in HEK293 cells; FIG. 2b) Rac1 activation and FIG. 2c) Cdc42 activation in the mutant cells relative to that observed in ARHGEF6 wt cells reveals the ARHGEF6 mutations identified in CKD patients selectively activates Rac1.

FIG. 3 Structural analysis indicates that the mutations of ARHGEF6 when superimposed on the crystal structure of Tiam1 cluster in a specific region of the DH domain and that they have close proximity to the interface residues that are distinct to Rac1 over Cdc42. Two projections are presented the right hand image is rotated by 90° relative to the left hand image as indicated in the figure. The position of the ARHGEF6 GI mutants projected onto the Tiam1 structure is indicated in Red. The Rac1 interface residues that are distinct relative the Cdc42 protein sequence are indicated in yellow. Rac1 is indicated in green (at front in left hand image, at top in right hand image), Tiam1 DH is indicated in blue (at back in left hand image, at bottom in right hand image).

FIG. 4: FIG. 4a) ARHGEF6 expression is higher in the glomeruli vs the tubulointerstitium, furthermore ARHGEF6 is upregulated in CKD glomeruli; FIG. 4b) ARHGEF6 upregulation is seen to emerge at CKD stage 1 and is maintained through to CKD stage 5, lower levels of ARHGEF6 in tubulointerstitium, with upregulation appearing from CKD stage 2 onwards; FIG. 4c) Glomerular ARHGEF6 was observed to be up-regulated to a statistically significant level across multiple CKD aetiologies.

FIG. 5: FIG. 5a) ARHGEF6 expression in BTBR ob/ob mice relative to control (C—BTBR lean mice) at 8, 24 and 20 weeks (normal diet) and at 14 and 20 weeks (with high protein (HP) diet) in the cortex and glomeruli reveals progressive upregulation in the glomeruli, but not tubuli, of BTBR ob/ob mice as time progresses; In situ hybridisation of samples from FIG. 5b) these mice and FIG. 5c) advanced diabetic nephropathy and FSGS (focal segmental glomerulosclerosis) patients revealed that ARHGEF6 expression is enriched in glomeruli (dark brown dots are ARHGEF6 signals).

FIG. 6: Effects of overexpression of ARHGEF6 in podocytes grown in culture: FIG. 6a) ARHGEF6 overexpression is seen to cause podocyte detachment; FIG. 6b) the CKD stressor PAN is seen to promote ARHGEF6 expression in cultured podocytes; FIG. 6c) depletion of ARHGEF6 in podocytes by lentiviral knockdown is observed to protect from PAN stimulated detachment.

FIG. 7: Depletion of ARHGEF6 in podocytes by lentiviral knockdown protects from PAN stimulated detachment by stabilising active beta-1 integrins.

FIG. 8: Partial depletion of ARHGEF6 is sufficient to rescue the podocyte phenotype stressed by PAN treatment: Image at top of figure: PAN treatment disrupts the organization of the actin cytoskeleton stress fibers while depletion of ARHGEF6 to various extents rescued stress fibres in both untreated and PAN-treated podocytes; Bottom left of figure: PAN treatment deactivates beta-1 integrins while depletion of ARHGEF6 to various extents rescued the activation of beta-1 integrins in both untreated and PAN-treated podocytes.; Bottom right of figure: active b1 integrins promote attachment to the GBM.

FIG. 9: ARHGEF6 gene and transcripts and alignment of ASO sequences. Three ARHGEF6 transcripts ENST00000250617, ENST00000370622 and ENST00000370620 (available at ensemble.org) are depicted with boxes indicating exons, connected by a line with arrows indicating introns. Coding sequences in exons are depicted by wider boxes and non-coding sequences are depicted by narrower boxes. Locations where ASOs 1, 2 & 3 described in Table 1 have perfect complementary homology are indicated by bars (ASOs row of figure).

FIG. 10: The role of ARHGEF6 upregulation in chronic kidney disease. The experiments detailed herein reveal that excess ARHEGF6 activity can be detrimental to both the podocyte cytoskeleton and levels of active beta-1 integrin. Excess ARHGEF6 is detrimental to normal kidney function as it compromises the actin cytoskeleton required to maintain podocyte morphology and thus the filtration slits between adjacent, tightly clustered podocytes on the glomerular filtration barrier and also, by inhibiting activation of beta-1 integrins required to maintain attachment if podocytes to the glomerular basement membrane. Experiments herein demonstrate that ARHGEF6 interacts with the client proteins PAK1 and GIT.

FIG. 11: ARHGEF6 overexpression induces apoptosis in human primary glomerular endothelial cells (HGMEC): FIG. 11a) HGMEC were transfected with either GFP or ARHGEF6 plasmids by electroporation. Twenty four hours after seeding the cells, annexin V fluorescence staining was monitored by live imaging for another 24 hours and ratio of annexin V positive cells was calculated. The % of Annexin V-positive cells relative to the total number of cells is higher in ARHGEF6 transfected cells than in GFP transfected cells. FIG. 11b) HGMEC were lysed after annexin V live imaging. Caspase 3/7 activity was measured in transfected HGMECs. Unpaired t-test, *P<0.05, ***P<0.001, ****P<0.0001.

FIG. 12: Incubation of THP-1 cells with the ARHGEF-6 ASO SEQ ID NO. 7 (TACAGTTTTCTTGGTC) reveals i) that THP-1 cell viability is maintained on long term (91h) exposure to ASO (CellTiterGlo®) and ii) knockdown of ARHGEF-6 (AlphaLISA®).

FIG. 13: Schematic Study Outline for the BTBR ob/ob mouse study. Mice are randomised at 7 weeks of age at which time baseline blood glucose and uACR levels were measured. IP dosing of control and ASO 9 at two doses levels commenced at week 9 and continued on a once weekly basis until week 20 (12 doses in total). Urine samples are taken periodically throughout the study to examine the evolution of uACR levels. Blood was taken at the end of study for further analysis.

FIG. 14: Downregulation of ARHGEF6 gene and protein expression following treatment with ASO 9. Left hand plot, ARHGEF6 gene expression relative to Hprt. Right hand plot ARHGEF6 protein expression (fmol/g). In each case, working from LHS to RHS, plots show result obtained in BTBR wt (healthy control), BTBR ob/ob (disease control) and BTBR ob/ob mice treated with ASO 9 at doses of 1 mg/mg and 8 mg/kg. Results presented as Mean±SEM. One-way ANOVA. ****=P-value<0.0001.

FIG. 15: A plot of estimated UACR means vs weeks on treatment as measured in the urine collected from BTBR ob/ob disease control treated with PBS (phosphate buffered saline, open circle) and groups treated with ASO 9 at a dose of either 1 mg/kg (triangle) or 8 mg/kg (solid circle). A statistically significant 61% improvement of renal damage was observed at 8 weeks of treatment for the 8 mg/kg ASO 9 cohort and maintained out to the end of study. The analysis is made with a model of the UACR data with treatment group and time as fixed effects using a mixed model for the repeated measurements for each subject over time. Post-hoc contrasts with the control group were made using Dunnett's method. ***=P-value<0.001 between BTBR ob/ob, control PBS, and BTBR ob/ob, ASO 9, 8 mg/kg.

FIG. 16: Glomerular score at the end of the 12-week ASO 9 treatment period based on data analysis using a mixed linear model (MLL), Student's t-test with Satterthwaite's method. Briefly, a trained AI algorithm is used to assign a numerical score to each glomerulus in an entire histological kidney section, providing an indication of the extent of glomerular damage. The definitions are 0: Normal; No or minimal changes, 1: Mild; slight-moderate mesangial matrix expansion with <4 mesangial cells/glomerular segment, 2: Moderate; moderate mesangial matrix expansion with 4-6 mesangial cells/glomerular segment, 3: Severe; moderate to severe mesangial matrix expansion with >6 mesangial cells/glomerular segment.

FIG. 17: FIG. 17A) Super resolution microscopy at the end of the 12-week ASO 9 treatment period. Working from left to right the picture shows podocyte staining in BTBR wt (healthy control), BTBR ob/ob (disease control) and BTBR ob/ob treated with ASO 9. First row images (Pictures A) and second row images (Pictures B) depict 2 different areas in the kidney glomeruli, showing the podocyte foot process. FIG. 17B) Results are presented as means in the plots of filtration slit density (FSD, left hand plot) and estimated glomerular diameter (right hand plot). Statistics—one-way ANOVA. **=P-value<0.01, ****=P-value<0.0001.

FIG. 18: Super resolution microscopy at the end of the 12-week ASO 9 treatment period. Working from left to right the Figure shows podocyte staining in BTBR wt (healthy control), BTBR ob/ob (disease control) and BTBR ob/ob treated with ASO 9 BTBR wt, BTBR ob/ob disease control and BTBR ob/ob treated with ASO 9. Images in top row (Pictures labelled A) and bottom row (Pictures labelled B) represent two different areas in the kidney glomeruli and arrows point to the green labelled endothelium (stained with EHD3).

FIG. 19: Plasma AST and ALT at the end of the 12-week ASO 9 treatment period. The levels of aspartate aminotransferase (AST) and alanine aminotransferase (ALT), the two liver enzymes used in healthcare as a biomarker for liver damage were not increased with ASO 9 treatment compared to BTBR ob/ob disease control mice over the course of treatment. Results are presented as Mean±SEM. One-way ANOVA and Dunnett's multiple comparison test were used to assign significance.

FIG. 20: Body weight, and organ weights at the end of the 12-week ASO 9 treatment period. Treatment with ASO 9 at doses of 1 mg/kg and 8 mg/kg did not have any effect in body weight or organ weight relative to disease control. Results presented as Mean±SEM. One-way ANOVA and Dunnett's multiple comparison test were used.

FIG. 21: ASO 9 exposure at the end of the 12-week ASO 9 treatment period. Concentrations of ASO 9 in the kidney, liver, skeletal muscle and heart of animals following 12 weeks of treatment at the 1 mg/kg and 8 mg/kg doses. Results are presented as mean±SEM. significances between the groups calculated by Students t-test. ****=P-value<0.0001.

### DETAILED DESCRIPTION

As noted above the present specification provides a method of treatment or prophylaxis of chronic kidney disease comprising administration of an inhibitor of ARHGEF6 activity to a patient in need thereof. The inhibitor of ARHGEF6 activity may act by reducing expression of ARHGEF6 protein, as is the case with an anti-sense oligonucleotide (ASO) inhibitor that binds to the mRNA produced by ARHGEF6 or with lentiviral knockdown, or can directly degrade ARHGEF6 protein, as is the case with a proteolysis targeting chimera (PROTAC) or with a small molecule degrader (i.e. a small molecule that causes degradation of ARHGEF6).

Identification of ARHGEF6 as a novel target for therapy or prophylaxis of chronic kidney disease and how precisely therapeutic entities for addressing this target should act is built on a series of studies inspired by an initial insight gained from a genomic analysis of samples obtained from CKD patients.

In more detail, a recent genomic study looking at rare variant associations for renal function and CKD involved whole exome sequencing of 3,150 kidney patient samples, encompassing diverse CKD subtypes, and 9,563 control samples. The proportion of cases and controls carrying rare variant(s) per gene were compared by a rare variant collapsing analysis approach with the aim of evaluating the contribution of rare variants to CKD risk in a large multi-ethnic population (Cameron-Christie et al, J Am Soc Nephrol. 2019. 30(6):1109-1122). As is noted in the Cameron-Christie paper, the relevance of rare variants can have broader implications. In more detail, detection of rare independent variants clustered in a single gene can provide significant insight into disease biology and influence clinical therapy in several ways, even if pathogenic variants in a particular gene only explain a small proportion of all cases. First, in a population with both acquired, inherited and multifactorial disease, accurate estimates of the proportion of cases caused by known genes can inform the use of existing therapies and diagnostic tests. Secondly, rare mutations can lead to the identification of widely applicable therapeutic drug targets, such as the discovery of PCSK9 mutations leading to the development of treatment for general forms of hypercholesterolemia. Thirdly, it is increasingly recognised that the validation of such drug targets in genetic studies of human populations improves the probability of the success of drug development in clinical trials. ARHGEF6 was one of a set of ca 300 genes with potential rare variant association to CKD/ESRD identified in this study, albeit no further link or significance of ARHGEF6 in CKD was provided or suggested.

In light of the results presented in the Cameron-Christie paper, and aware of the role of ARHGEF6 in cytoskeletal rearrangement and motility processes and, further, that separate studies had suggested that hyperactivation of Rac1, a GTPase that binds to, and is activated by, ARHGEF6, was associated with kidney damage, we were drawn to explore whether a causative link between ARGHEF6 activity and CKD could be established. Furthermore, if such a causative link could be established, we wondered whether direct or downstream inhibition of ARHGEF6 activity might provide a new opportunity for the treatment and/or prophylaxis of CKD. Should inhibition of ARHGEF6 activity provide such an opportunity for treatment or prophylaxis of CKD, we sought to understand what characteristics an effective inhibitor of ARHGEF6 activity should possess. As detailed below, the results from the investigations detailed herein demonstrate that effective treatment of CKD will require modulation of ARHGEF6 protein activity, for example at the protein level via downregulation of ARHGEF protein expression (e.g. with an anti-sense oligonucleotide) or by causing degradation of ARHGEF6 protein levels (e.g. with a PROTAC or small molecule) and that merely blocking the interaction of the ARHGEF6 with further proteins, directly or indirectly, such as Rac1, to block downstream effects may not be optimal.

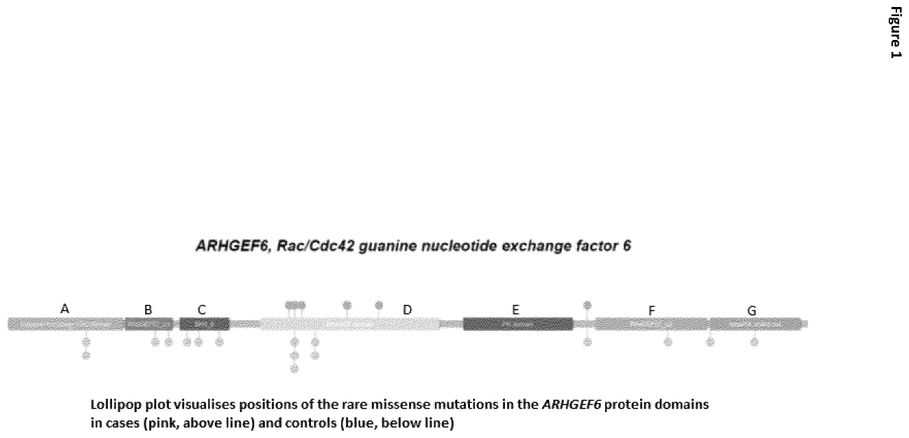

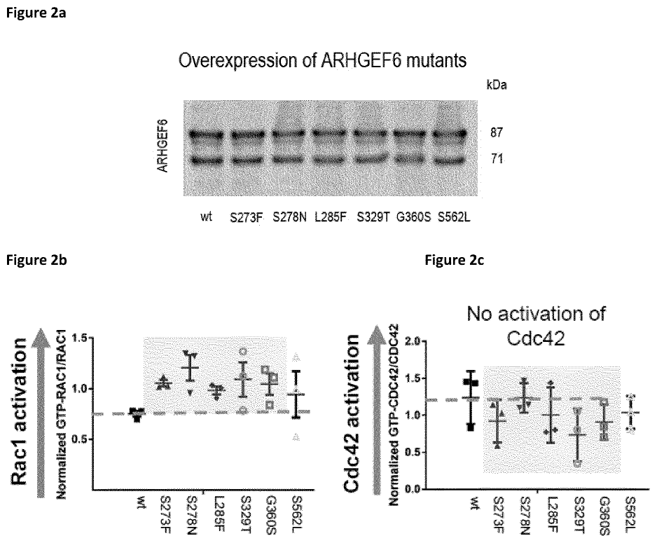

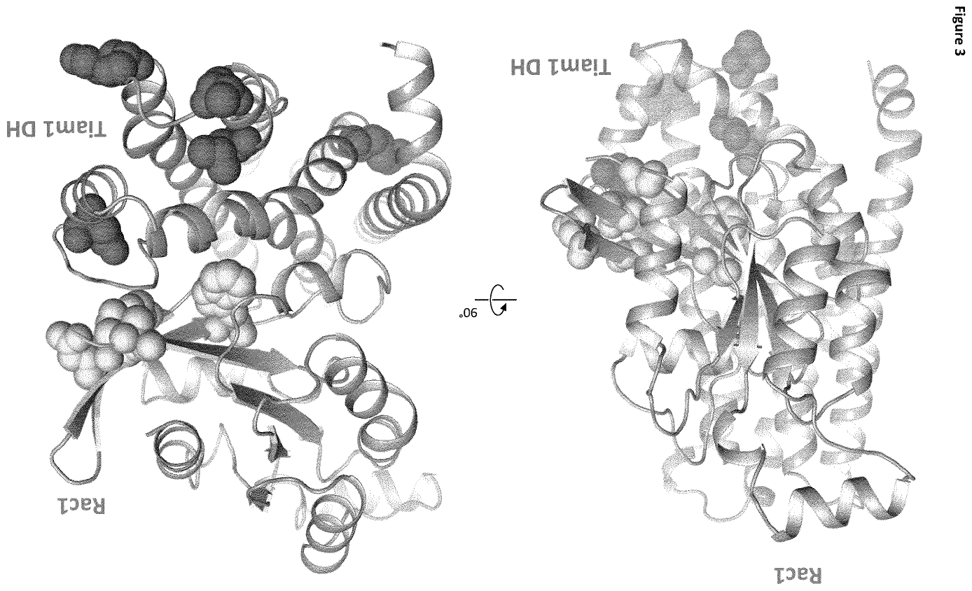

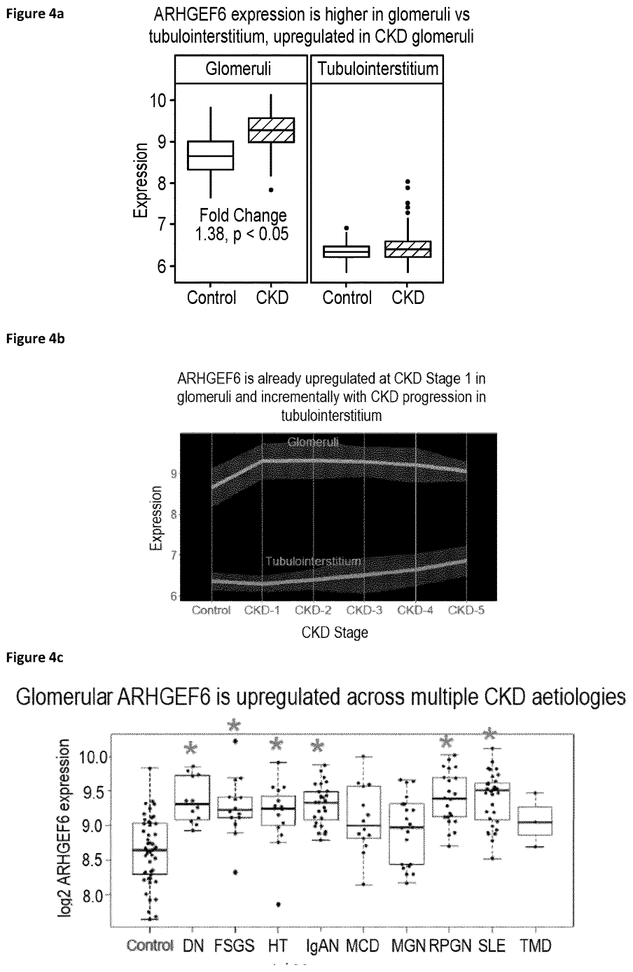

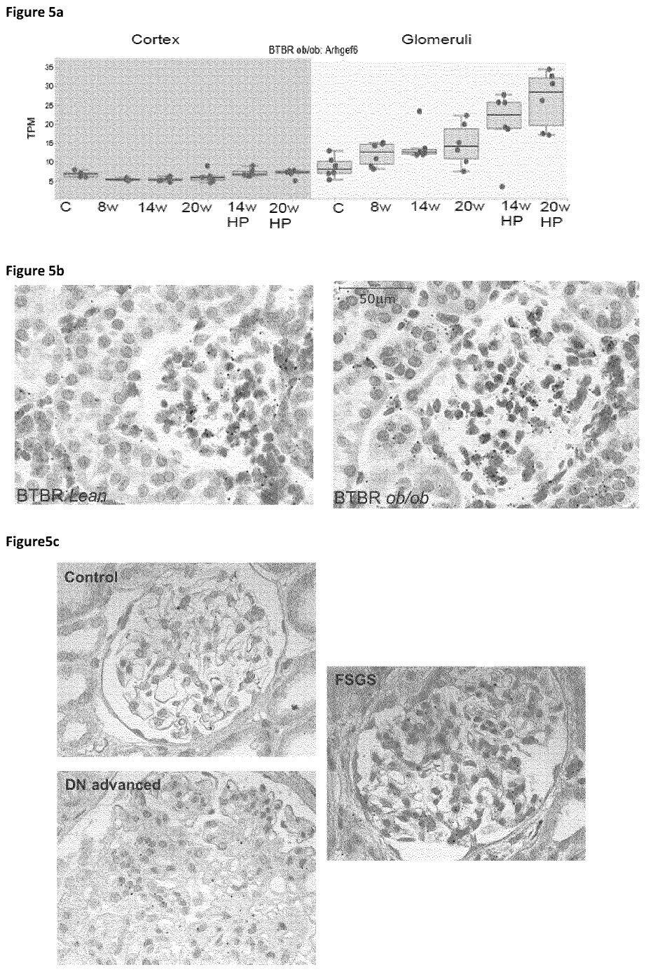

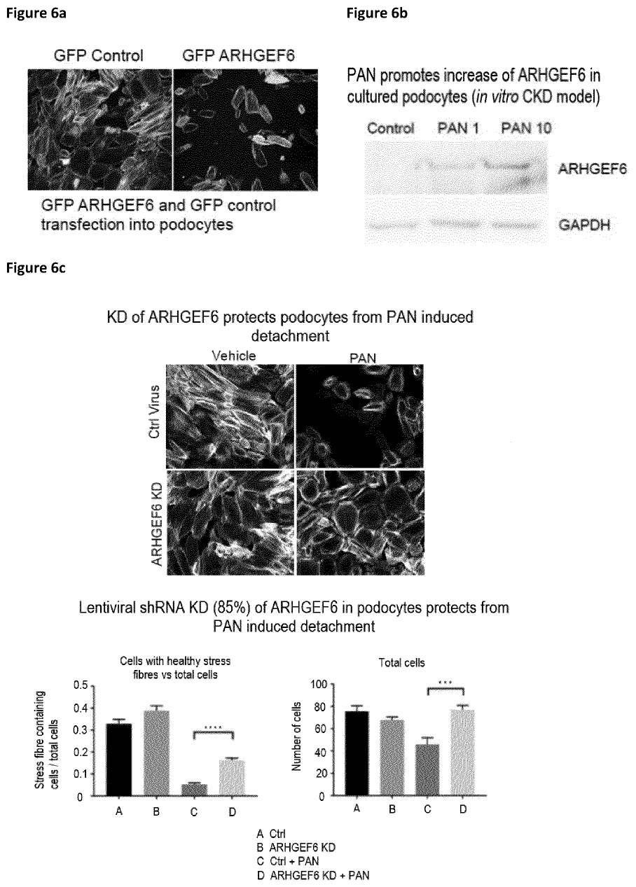

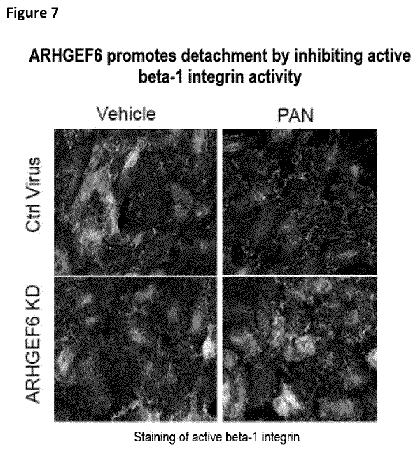

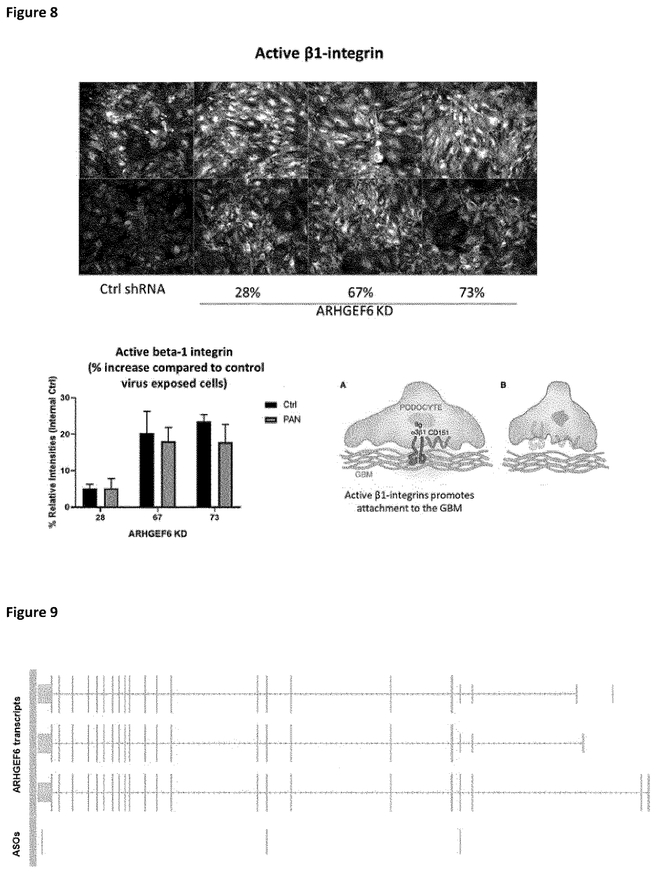

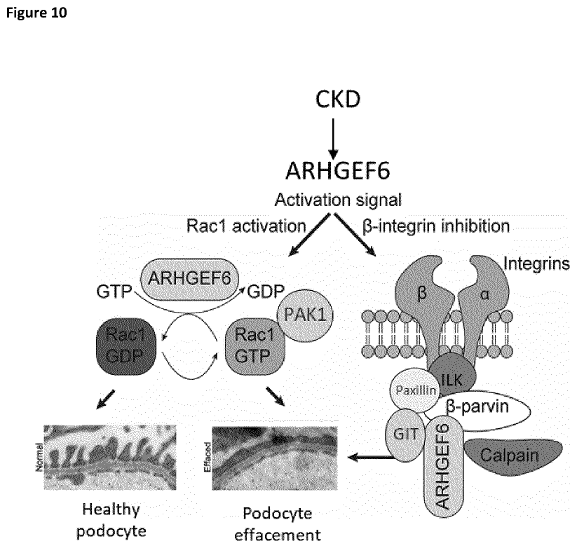

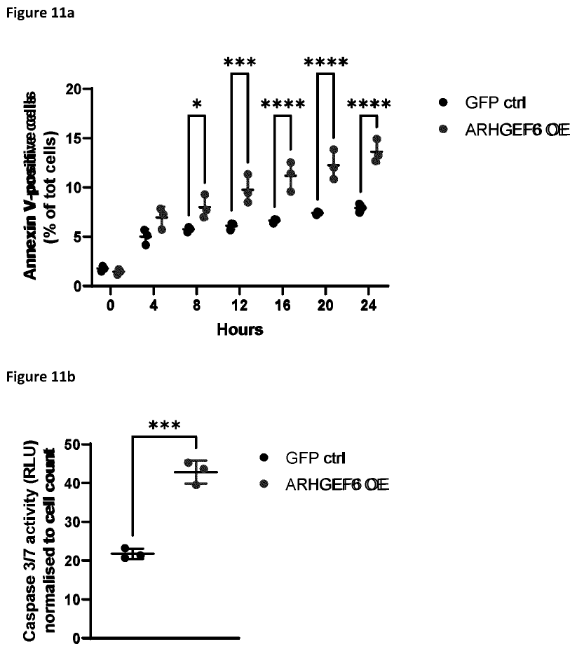

The experiments that we performed while seeking to establish a link between ARGHEF6 and CKD are described in full detail in the examples section below. A synopsis of the results obtained from this research and their significance are summarised in i) to viii) below:
i) The location of the ARHGEF6 mutations identified in the study reported by Cameron Christie et al were projected on a Lollipop plot to determine if there was any particular association in the sites of mutation. The resultant plot revealed that five of six observed rare variant ARHGEF6 mutations identified in the study were located in the DH downstream effector domain thought to be responsible for Rac1 binding and GEF catalytic activity (see FIG. 1). This clustering of rare variant mutations encouraged further investigation into what the functional consequences of these mutations might be.
ii) CRISPR knockdown of ARHGEF6 in HEK293 cells followed by transfection with both wild type (wt) and mutated ARHGEF6 was used to generate HEK293 derived cells overexpressing wt ARHGEF6 or ARHGEF6 with the point mutations identified in the rare variants genomics analysis (S273F, S278N, L285F S329T, G360S and S562L) (FIG. 2a). As can be seen from FIGS. 2b and 2c, increased Rac1 activation was observed in all six cell lines overexpressing mutated ARHGEF6 relative to the HEK cells overexpressing wt protein, while no significant effect on Cdc42 activation could be observed. The mutations of ARGHEF6 identified in the rare variant genomics analysis were thus identified as gain of function mutations that selectively activate Rac1, but not Cdc42.
iii) As an X-ray protein crystal structure for ARHGEF6 itself was not available, we were drawn to inspect the X-ray crystal structure of the closest homolog (Tiam1) for which a structure was available to see where the mutated residues were located. Previous crystallographic studies had revealed that the Tiam1-Rac1 interaction involves a relatively large surface area (over 3000 Å²) centred on the DH domain of Tiam1 (Worthylake et al, Nature 2000, p 682-688). In this structure, the PH domain of Tiam1 appears to stabilise the DH domain so that it can efficiently bind to Rac1. When the residues of ARGHEF6 found to be mutated in the rare variant genomics analysis of CKD patients were projected onto the Tiam1 structure (see FIG. 3), they are seen to be clustered around the α5 and α6 helices of the DH domain that interact with Rac1 (and not Cdc42).
iv) Human transcriptomic analysis of ARGHEF6 revealed that ARHGEF6 expression is significantly higher in the glomeruli relative to the tubulointerstitium and that expression of ARHGEF6 in CKD patients is higher than in control (1.38 fold change, p<0.05) (FIG. 4a). A significant increase in ARHGEF6 expression in the glomeruli was observed to occur in CKD stage 1, i.e. at the early stage of CKD, and this expression proved to be maintained across the later stages of the disease (FIG. 4b). A more progressive, lower level, upregulation of ARHGEF6 in the tubulointerstitium was seen in the same analysis—in this case however upregulation appears to initiate at CKD stages 2/3. This data supports the postulate that ARHGEF6 activity could be a causative factor in CKD due to the early and sustained upregulation from the earliest stage of the disease. Glomerular ARHGEF6 was observed to be up-regulated to a statistically significant level across multiple CKD aetiologies including DN (diabetic nephropathy), FSGS (focal segmental glomerulosclerosis), HTN (renal hypertension), IgAN (IgA nephropathy/Berger's disease), RPGN (rapidly progressive glomerulonephritis) and SLE (systemic lupus erythematosus) but not MCD (minimal change disease), MGN (membranous glomerulonephritis) and TMD (thin glomerular basement membrane disease).
v) As ARGHEF6 expression is not localised in the podocytes of the kidney alone, we sought to establish whether there were signs of selective ARGHEF6 upregulation in podocytes in an obese mouse model of CKD diabetic nephropathy (the BTBR ob/ob mouse model described in Hudkins et al, J Am Soc Nephrol 2010, 21(9), p 1533-42). The BTBR ob/ob mice spontaneously develop diabetes at 6 and 8 weeks (male/female respectively). Compared to control, BTBR lean, mice transcriptomic analysis revealed that ARHGEF6 upregulation in obese mice increased over time in the glomeruli, but not in the cortex (FIG. 5a). In situ hybridisation of samples from these mice (FIG. 5b) and advanced diabetic nephropathy and FSGS patients (FIG. 5c) revealed that ARHGEF6 expression is enriched in glomeruli (the small dark brown dots in the image are ARHGEF6 signals) and not tubuli. A correlation between upregulation of ARHGEF6 and the development of CKD diabetic nephropathy and focal segmental glomerulosclerosis is seen and this is in agreement with the human transcriptomic analysis described in iv) above.
vi) ARHGEF6 overexpression in cultured podocytes was observed to cause detachment of the podocytes from culture (cells transfected with green fluorescent protein (GFP) used as a control did not detach (see FIG. 6a)). In complimentary experiments, exposure of cultured podocytes to the CKD stressor PAN was found to induce ARGHEF6 expression (FIG. 6b) and promote detachment of podocytes from culture (FIG. 6c). The PAN promoted detachment of podocytes did not occur when ARHGEF6 function was inhibited by lentiviral shRNA knockdown of ARHGEF6 (FIG. 6c), demonstrating that inhibition of ARHGEF6 activity, via suppression of ARHGEF6 at the protein level, is protective against podocyte cytoskeletal rearrangement and that ARHGEF6 activity also promotes detachment from cell culture.
vii) An insight into the mechanism by which ARHGEF6 promotes detachment was provided by staining PAN stressed podocytes treated with lentiviral shRNA for ARHGEF6 and control for active beta-1 integrins ((β1-integrins). (β1-integrin expression by podocytes has been demonstrated to be essential for maintaining the structural integrity of the glomerular filtration barrier as active β1-integrins are essential for adhesion of the podocytes to the glomerular basement membrane (see Pozzi et al, Developmental Biology 2008, (316), p288-30). As can be seen from FIG. 7, inhibition of ARHGEF6 activity by lentiviral shRNA knockdown, preserved the active (β1-integrins important for glomerular structural integrity and consequently protects podocytes from PAN induced detachment in culture. Thus counteracting the induction of ARHGEF6 protein, that otherwise would have resulted from exposure to the CKD stressor PAN, was shown to protect the glomerular filtration barrier by maintaining both the cytoskeletal integrity of podocytes on the GBM (maintenance of the integrity of the podocytes' actin cytoskeleton) and the attachment of podocytes to the GBM (maintenance of active beta-1 integrin levels).
viii) To further dissect the mechanism by which ARHGEF6 promotes detachment of podocytes and to complement the observations above that suggest that inhibiting the ARHGEF6 mediated activation of Rac1 in isolation would not fully abrogate the detrimental effects of ARHGEF6 upregulation in CKD an experiment was performed to investigate the effect of partial knockdown of ARHGEF6. This experiment was designed to establish whether partial knockdown of ARHGEF6 would protect against the deleterious effects on podocytes we had established results from ARHGEF6 upregulation. A titration experiment was thus performed in which PAN stressed podocytes were treated with varying amounts of lentiviral shRNA for ARHGEF6 to reduce ARHGEF6 protein levels stimulated by PAN. Results from this experiment indicated that a partial depletion of ARHGEF6 protein was sufficient to restore levels of active (β1-integrins to the levels observed in normal, non-PAN stressed, podocytes (FIG. 8). Modulation of the ARHGEF6 protein levels is therefore established as having a positive, restorative, benefit for the maintenance of the actin cytoskeleton of the podocytes on the glomerular filtration barrier, through down regulating ARHGEF6 mediated Rac1 activation to “normal” levels thus protecting the podocyte's actin cytoskeleton and, in addition, by promoting adhesion of podocytes to that barrier through restoring “normal” active β1-integrin levels.
ix) In addition to podocyte injury, endothelial dysfunction is another hallmark of CKD. Multiple factors including oxidative stress, inflammation, advanced glycated end product and uremic toxins contribute to renal endothelial damage (see e.g. J. Malyszko, Clin Chim Acta. 2010 Oct. 9; 411(19-20):1412-20. doi: 10.1016/j.cca.2010.06.019). Experiments using single cell RNAseq (see e.g. T. Andrews, M. Hemberg, Molecular Aspects of Medicine 59 (2018), 114-122) and RNAscope (see e.g. https://acdbio.com/science/how-it-works., F. Wang et al, J Mol Diagnostics, 2012, 5(2):210-219), revealed that ARHGEF6 was expressed in glomerular endothelial cells in both human and mouse kidneys. To study the functional impact of ARHGEF6 upregulation in endothelial cells, ARHGEF6 was overexpressed by electroporation of ARHGEF6 plasmids in primary human glomerular endothelial cells (HGMEC). ARHGEF6 overexpression was found to significantly increase apoptosis of HGMEC as was evident from the significantly increase in Annex V positive HGMECs over time and increased caspase 3/7 activity in ARHGEF6-overexpressing compared to control plasmid-transfected HGMECs (FIGS. 11a and 11b, respectively). These results suggest that upregulated ARHGEF6 may partially contribute to endothelial dysfunction in CKD. Accordingly, taken together with the effects of ARHGEF6 overexpression on the podocyte cytoskeleton and their attachment to the GBM described above, the overexpression of ARHGEF6 is demonstrated to be detrimental to multiple components of the glomerular filtration barrier.
x) ASOs directed to human and mouse ARHGEF6 have been generated and profiling has demonstrated that highly potent, non-toxic ASOs can be generated. Furthermore, prolonged exposure of relevant cells to such ASOs, and high degrees of ARHGEF6 knock down in the same cells, is well tolerated thus supporting progression of this modality to clinical evaluation.

Taken together the results of the experiments described above establish that administration of an agent that can deplete or degrade ARHGEF6 protein levels to deliver a level associated with normally functioning kidneys can restore the cytoskeleton of podocytes and their attachment to the GBM. Consequently, a new method for the treatment of CKD has been established that involves administration of an inhibitor of ARHGEF6 activity to a patient in need thereof. The inhibitor of ARHGEF6 activity is preferably an agent that functions to reduce the ARHGEF6 mediated activation of Rac1 and that protects against inactivation of active β1 integrins. The inhibitor of ARHGEF6 activity, as demonstrated herein, can act to deplete protein expression or directly degrade ARHGEF6 at the protein level thereby favouring maintenance or restoration of a “normal” actin cytoskeleton in the podocytes on the GBM and the attachment of podocytes to the GBM via restoration of “normal” levels of activated β1-integrins. For example, the inhibitor of ARHGEF6 activity can act through downregulation of ARHGEF6 at a protein level as is the case with an ASO (as, for example, is demonstrated with lentiviral knockdown experiment described above and directly with ASOs below). Alternatively, the inhibitor of ARHGEF6 activity can act through degradation of ARHGEF6, as is the case of a proteolysis targeting chimera (a PROTAC) or a small molecule degrader.

To further prove the concept set forth above, and foreshadowed at x) above, a set of antisense oligonucleotides (ASOs) to ARHGEF6 were designed, synthesised and evaluated for their ability to reduce ARHGEF6 expression in human THP-1 cells. ASOs with a length of 16 nucleotides were designed against the ARHGEF6 (ENSG00000129675) gene sequence and its unspliced transcripts. Regions accessible to ASO targeting were identified using the Vienna RNA RNAplfold algorithm (see Bernhart, S. H., Muckstein, U. & Hofacker, I. L. RNA Accessibility in cubic time. Algorithms Mol Biol 6, 3 (2011)), with preliminary design of ASOs performed following the general guidelines set forth in Oligonucleotide-Based Therapies: Methods and Protocols. (Humana Press, 2019). doi:10.1007/978-1-4939-9670-4. Candidate ASOs (complement base pair sequences) targeting the accessible regions were then further filtered prior to synthesis based to satisfy criteria a) to f) below:
a) accessibility score>0.001 (using the Vienna RNA RNAplfold algorithm);
b) perfect complementarity to only the ARHGEF6 sequence;
c) complementarity with 1 mismatch to no greater than 50 other genes;
d) ASOs do not target a region with a minor allele frequency>0.05,
e) no CG motifs; and

$$\% GC > 10 ; \qquad f )$$

ASO designs were further prioritised on the basis of their predicted lack of propensity for duplex formation with itself (i.e. another identical ASO) and low likelihood of self-folding thereby ensuring that the ASO present in a conformation suitable for bind to the target, ARHGEF6, mRNA (algorithms for determining these factors are provided in numerous sources e.g. Lorenz, R., Hofacker, I. L. & Stadler, P. F. RNA folding with hard and soft constraints. Algorithms for Molecular Biology 11, 8 (2016)).

A first set of 50 ASOs were randomly selected for synthesis out of the resultant, filtered, design set of ASOs meeting criteria a) to f) to evenly cover the ARHGEF6 transcripts. ASOs were synthesized by standard automated RNA synthesis as 3-10-3 gapmers with a LNA-DNA-LNA structure. A gapmer is a chimeric antisense oligonucleotide that contains a central block of deoxynucleotide monomers (i.e. a unit of DNA) sufficiently long to induce RNase H cleavage. The component ribonucleic acid monomer units in the overall oligonucleotide are connected by phosphate linkages between the 3′-O and 5′-O ribose groups of adjacent ribonucleic acid monomers (delivering a phosphodiester linkage). A LNA-DNA-LNA gapmer is a gapmer that features LNA monomers at the 3′- and 5′-ends of a central DNA unit. LNA refers to locked nucleic acid monomers—modified A, C (or 5-Me-C), G or T ribonucleic acid monomers in which the ribose moiety is modified with a methylene bridge connecting the 2′- and 4′-carbons of the ribose ring. A 3-10-3 LNA-DNA-LNA gapmer is therefore an oligonucleotide containing a central unit of 10 A, C (or 5-Me-C), G or T deoxynucleotide monomers (nucleotides featuring 2-deoxyribose moieties) flanked by units containing 3 LNA monomers at the respective 3′-, and 5′-termini, of the overall ASO sequence. Replacement of C deoxynucleotide monomers with 5-methyl deoxycytosine (5-Me-dC) can be advantageous in ASOs for prevention or reduction of undesirable immune responses. The ASOs described herein feature 5-Me-C LNA and DNA units (as indicated by mC in Table 1).

The advantages of using a LNA-DNA-LNA gapmer design are well known in the field. Briefly, the methylene bridge in the LNA fixes or “locks” the ribose motif in a 3′-endo conformation resulting in a reduction of conformational flexibility of the ribose and an increase in the degree of local organization of the phosphate backbone. This entropic constraint leads to improved binding to complementary RNA and DNA sequences among other advantages (see e.g. Elayadi et al, Biochemistry 2002, 41(31), 9973-9981. A number of synthetic ribonucleic acid derivatives featuring a bridge connecting the 2′- and 4′-carbons of the ribose ring are available and are known collectively as bridged nucleic acids (BNAs). Binding of the ASO gapmer to its complementary target mRNA causes RNase recruitment followed by mRNA cleavage by RNase, causing a downregulation of gene expression. The mRNA cleavage releases the ASO gapmer allowing it to function in a catalytic manner.

The library of synthesized ASO were evaluated for their ability to reduce ARHGEF6 expression in THP1 cells. Results from 3 exemplary ASOs, SEQ ID NOS 1, 2 and 3 along with the knockdown of ARHGEF6 in THP1 cells are presented in Table 1. The distribution of the ASOs across the ARHGEF6 transcripts is shown in FIG. 9 (ASOs 1, 2 and 3 are ordered left to right in the Figure).

TABLE 1
Human ARHGEF6 ASO sequences and ARHGEF6 knockdown (%) in THP1
cells (all 3-10-3 LNA-DNA-LNA gapmers, C denotes 5-Me-C for both
LNA and DNA constituents, LNA components in bold). Potency
figures are provided as the negative log of the IC50 value when
converted to molar.

| | | ARHGEF6 | |
|---|---|---|---|
| | | knockdown | Potency |
| ASO # | Oligonucleotide sequence of ASO | (%) | (pIC50) |
| 1 | GTATTAGCAGCACTAA (SEQ ID NO: 1) | 47.1 | |
| 2 | TGTACCATGTAGCTAT (SEQ ID NO: 2) | 39.5 | |
| 3 | AATGTGCATACTTCCT (SEQ ID NO: 3) | 52.0 | |
| 4 | GATGGTTTGCACAGTA (SEQ ID NO. 4) | 85 | 6.4 |
| 5 | GGTTTGCACAGTAAGC (SEQ ID NO. 5) | 85 | 7.0 |
| 6 | GGATGGTTTGCACAGT (SEQ ID NO. 6) | 92 | 7.1 |
| 7 | TACAGTTTTCTTGGTC (SEQ ID NO. 7) | 77 | 6.6 |

As can be seen from Table 1, the prototype ASOs designed to target ARHGEF6 across the gene proved capable of reducing ARHGEF6 expression in cells. The ability of ASOs targeting ARHGEF6 to modulate ARHGEF6 expression is thus demonstrated and, taken with the link established between ARHGEF6 upregulation described herein, targeting of ARHGEF6 is shown to be a feasible modality for the treatment of chronic kidney disease.

To extend on these preliminary results we looked to obtain more active mouse and human ASOs. A set of prototype mouse ASOs were designed and synthesised according to the algorithm described above for SEQ ID NOS: 1 to 3 to give a set of molecules that proved capable of knocking down ARHGEF6 protein expression in murine cells. Generating mouse ASOs was desirable as it provides the opportunity to probe for efficacy, selectivity and toxicity in non-human model systems.

In order to optimise the degree of knockdown delivered by the prototype ASOs (both mouse and human) RNA walk processes were performed. The RNA walk process involves systematically shifting the targeted RNA sequence from that targeted by the initial prototype ASO by one or several bases. In practice this involves deleting one or more nucleoside from one end of the existing ASO and adding the same number of nucleosides to the opposite end of the ASO, with the newly added nucleosides being complementary to the corresponding mRNA sequence. The activity of four further, human, ASOs (SEQ ID NOS: 4 to 7) is presented in Table 1 which illustrates that highly potent and effective ASOs for human ARHGEF6 can be generated.

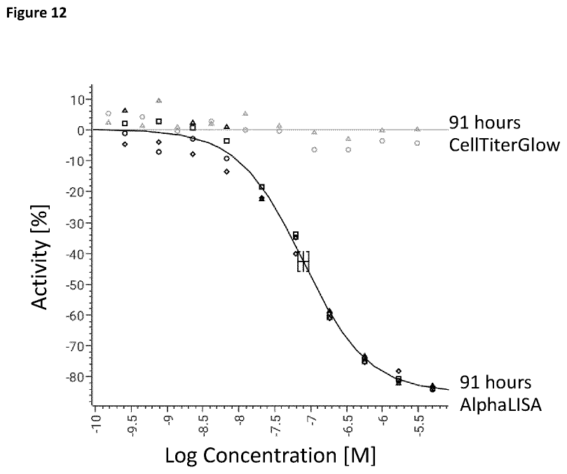

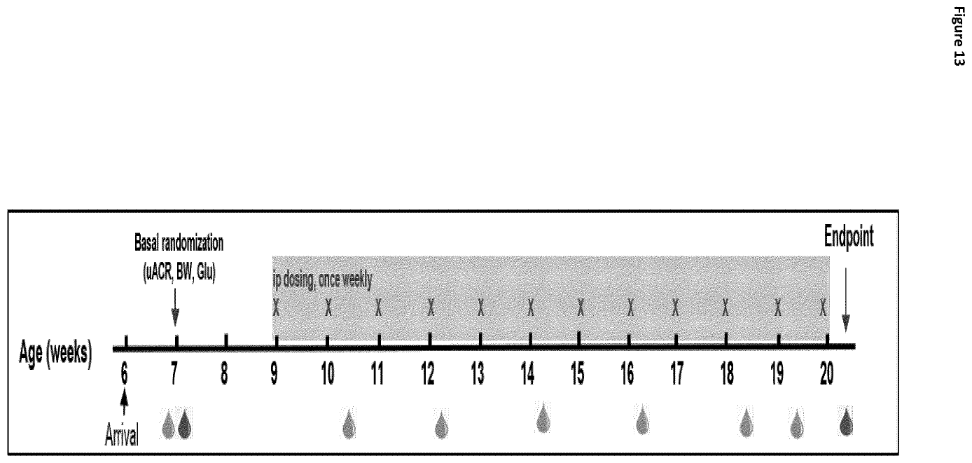

An experiment was performed to evaluate the effects of long term exposure of THP-1 cells to the ASO SEQ ID NO. 7 to establish whether exposure to the ASO or knockdown of ARHGEF-6 in the cells would compromise cell viability. Results of this experiment are presented in FIG. 12. In this experiment THP-1 cells were incubated with ASO SEQ ID NO. 7 for 91 hours. Replicate incubations for assessment of cell viability CellTiterGlo® (see e.g. Promega.co.uk for details of this luminescent cell viability assay that is based on the quantification of the ATP present, with ATP being used as an indicator of metabolically active cells) and an ARHGEF-6 AlphaLISA® (see perkinelemer.com for; an ARHGEF-6 specific AlphaLISA® was developed inhouse based on PerkinElmer Anti-Rabbit IgG AlphaLISA Acceptor Beads (AL104C), Anti-Mouse IgG Alpha Donor beads (AS104), ARHGEF6 purified MaxPab mouse polyclonal antibody (B01P) (Abnova, H00009459-B01P) and Cool2/aPix (C23D2) mAb monoclonal (Rabbit) antibody (CST, 4573S)), used to measure expression of ARHGEF-6 protein expression. The CellTiterGlo® assay and the AlphaLISA® assay were normalised to H₂O control. The AlphaLISA® assay was normalised to neutral control (H₂O) and inhibition control ASO that showed >90% ARHGEF-6 knockdown. As can be seen from FIG. 12 THP-1 cells could be grown for sustained periods in a plate with a volume of 10 L/well. No effect of ASO treatment on cell viability was seen over the course of the experiment (91 h incubation time), ARHGEF-6 knockdown over the same time course approached 100%.

A set of mouse ASOs that were generated following RNA walks, along with knockdown, cytotoxicity (Caspase MEC) and potency are presented in Table 2. As can be seen from Table 2, as set of highly active mouse ASOs were generated. Caspase MEC (minimum effective concentration in μM with a minimum threshold set to 30%) is a measure of the cytotoxicity of the ASO, with a mean EC30 value of >0.1 μM being considered to be indicative of an ASO with safe hepatotoxicity and cytotoxicity profile.

TABLE 2
Mouse ARHGEF6 ASO sequences and their activity in reducing ARHGEF6 expression in
Raw264.7 cells (ASOs are all 3-10-3 LNA-DNA-LNA gapmers, C denotes 5-Me-C for
both LNA and DNA constituents, LNA components in bold). Potency figures are
provided as the negative log of the IC50 value when converted to molar.

| | | ARHGEF6 | Caspase | Potency |
|---|---|---|---|---|
| ASO # | Oligonucleotide sequence | knockdown (%) | MEC (μM) | (pIC50) |
| 8 | GAATTTAGGTATACAG (SEQ ID NO: 8) | 96 | >3 | 5.8 |
| 9 | CCAATATAGTATTCTG (SEQ ID NO: 9) | 87 | >10 | 6.1 |
| 10 | AGTTATGTGTAGGATT (SEQ ID NO: 10) | 82 | >3 | 6.1 |
| 11 | GTTATGTGTAGGATTA (SEQ ID NO. 11) | 87 | 0.66 | 6.1 |
| 12 | GCAAATTTGTTGATAG (SEQ ID NO. 12) | 96 | 1.35 | 6.0 |
| 13 | GACATTTTACTTATCC (SEQ ID NO. 13) | 82 | 0.77 | 6.0 |
| 14 | CATGAATGGTTTGCTG (SEQ ID NO. 14) | 66 | 1.05 | 5.8 |
| 15 | ATTAGTTATGTGTAGG (SEQ ID NO. 15) | 86 | 0.14 | 6.1 |
| 16 | GTTATGTGTAGGATTA (SEQ ID NO. 16) | 86 | 0.78 | 6.1 |

The translation of this ASO activity in in vitro systems into the in vivo context is underway, as is optimisation of ASOs for therapeutic use in humans. Results of a first proof of concept study demonstrating that knock down of ARHGEF6 gene and protein levels can be used to restore renal filtration barrier function in CKD are reported below.

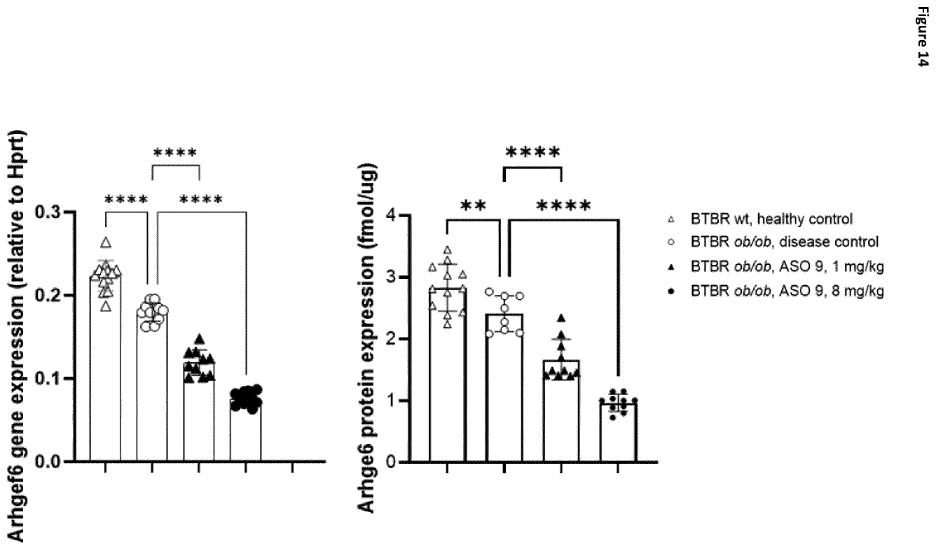

In order to obtain an in vivo proof of concept confirming that knock down of ARHGEF6 gene and protein levels could be used to restore renal filtration barrier function in CKD, a study was run to evaluate the impact of ARHGEF6 ASO administration to diabetic BTBR ob/ob mice. Functional readouts utilised to understand the effect on the filtration barrier include longitudinal urinary albumin to creatinine analysis in combination with histological and structural super resolution analysis of the glomeruli. A schematic outlining the study is presented as FIG. 13.

Leptin-deficient BTBR ob/ob mice (homozygous for the spontaneous mutation, Lep^{ob}) exhibit extreme obesity due to hyperphagia. They develop severe and progressive hyperglycemia and hypertriglyceridemia, elevated plasma insulin, impaired wound healing and are also hypometabolic and hypothermic (see e.g. K. L Hudkins et al, J Am Soc Nephrol 2010, Sep, 21(9), 1533-42). As such, the diabetic BTBR ob/ob mouse model mimics features of early diabetic neuropathy (DN) in humans (CKD/DN stage 2 in man), with chronic injury limited to glomeruli. These mice also develop severe progressive albumin and proteinuria and some of the morphological features typical for human DN, such as mesangial expansion, basement membrane thickening, and, to some extent, mesangiolysis. The BTBR ob/ob mice are hyperfiltrating, thereby resembling early stages of human DN.

As described in detail herein, the influence of intraperitoneally dosed ARHGEF6 ASO treatment vs control showed beneficial effects on restoration of the filtration barrier with less albumin leaking into the urine (proteinuria, UACR) as a result of the improved structural improvement on the filtration barrier obtained by the ASO.

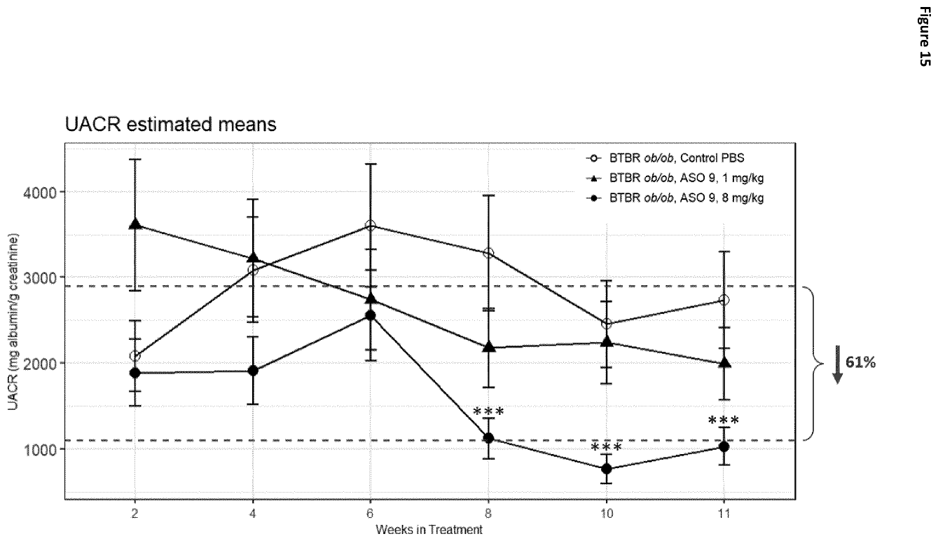

A first confirmation that intraperitoneal (ip) administration of the mouse ARHGEF6 ASO, ASO 9, can deliver a knock-down effect on ARHGEF6 gene and protein expression in mouse kidney is shown in FIG. 14. The extent of knock down was seen to increase in a dose dependent manner.

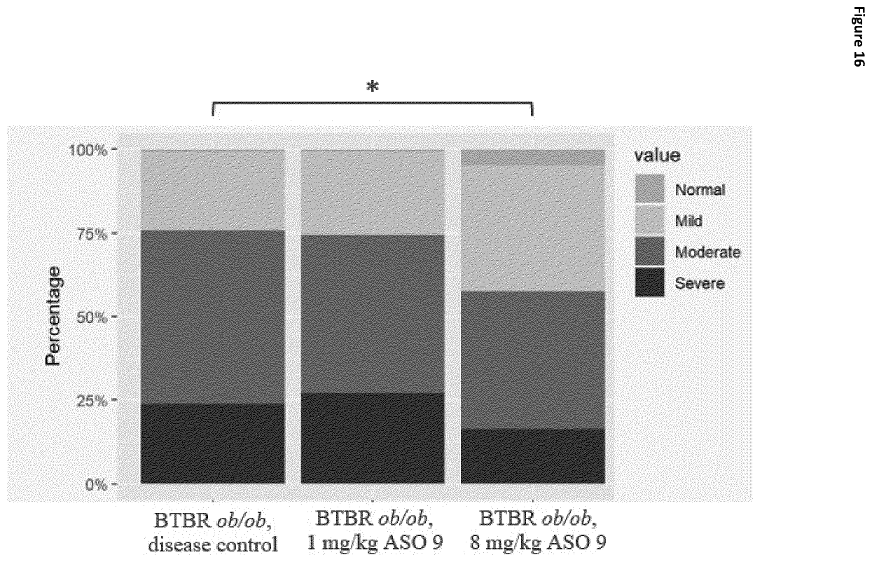

Furthermore, as can be seen from FIG. 15, once weekly ip administration of ASO 9 at 1 mg/kg and 8 mg/kg doses delivered promising reductions in UACR levels with a 61% reduction in UACR levels relative to control being obtained in the 8 mg/kg per week ASO 9 dose cohort. In addition, UACR levels in the 8 mg/week ASO 9 dosed cohort were seen to reduce over the course of the study relative to UACR levels at the outset of the study. Taken together, the data obtained in this study suggest that not only can ARHGEF6 ASO treatment protect against progression of kidney damage in the BTBR ob/ob mice model, it may also be possible that control of ARHGEF6 protein levels might potentially reverse kidney damage already sustained.

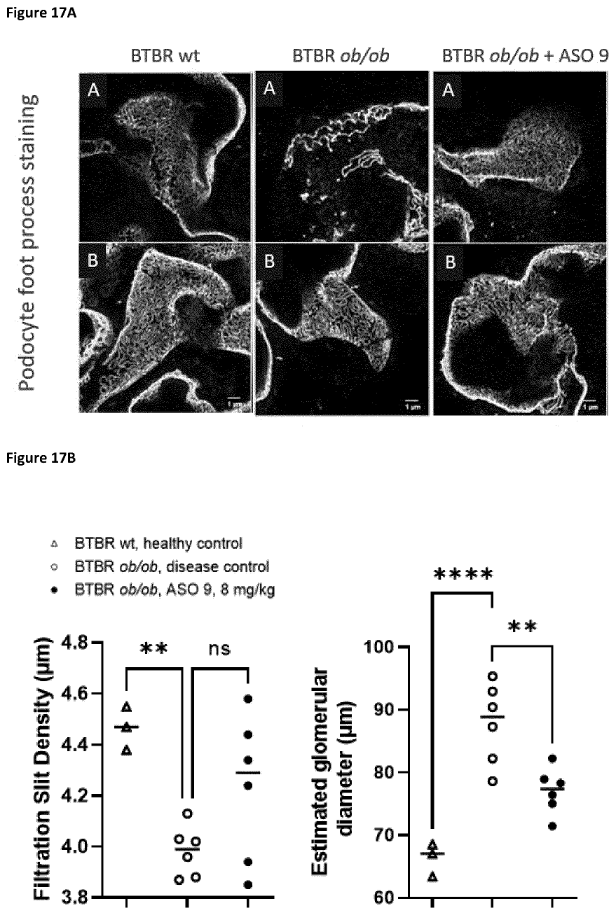

In addition to the promising effect on UACR, the 8 mg/kg per week ASO 9 dosed cohort, exhibited a significantly improved glomerular score as shown in FIG. 16. In the histological sections obtained in the study it was observed that the amount of severely injured glomeruli in ASO treated sections is significantly reduced, while the number of normal glomeruli is increased (there were almost non-existent normal glomeruli in the BTBR ob/ob control mice treated with placebo).

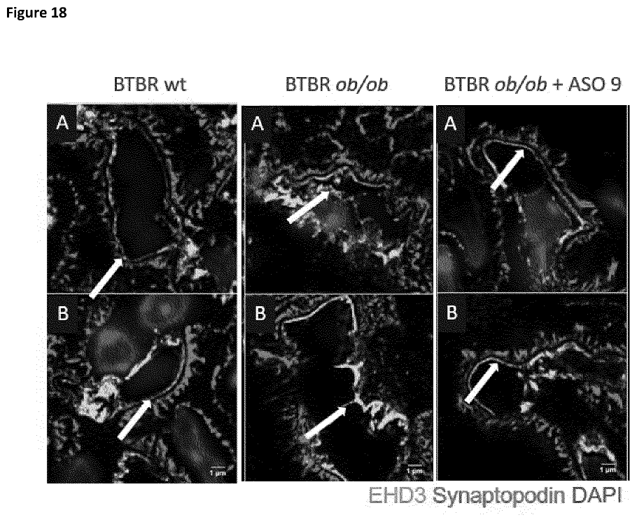

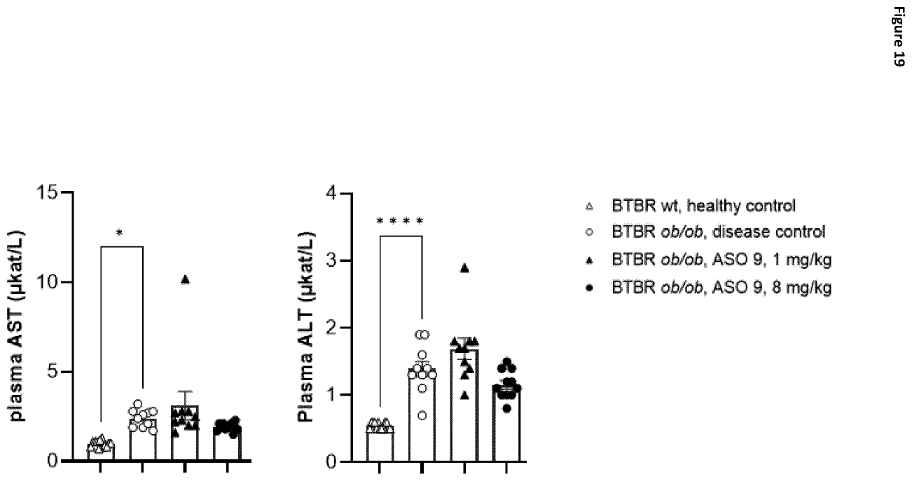

Improvements in glomerular health was also confirmed using super resolution microscopy. ASO 9 treatment resulted in repair of podocyte foot processes (as indicated by filtration slit density (FSD)) and restored glomerular size (as indicated by reduced glomerular diameter) as can be seen in FIG. 17. In addition, improved glomerular endothelial morphology was observed following ASO 9 treatment with the reduction of endothelial protrusions in BTBR ob/ob ASO treated samples resembling those in the healthy endothelial phenotype seen in BTBR wild type (wt) mice (see FIG. 18).

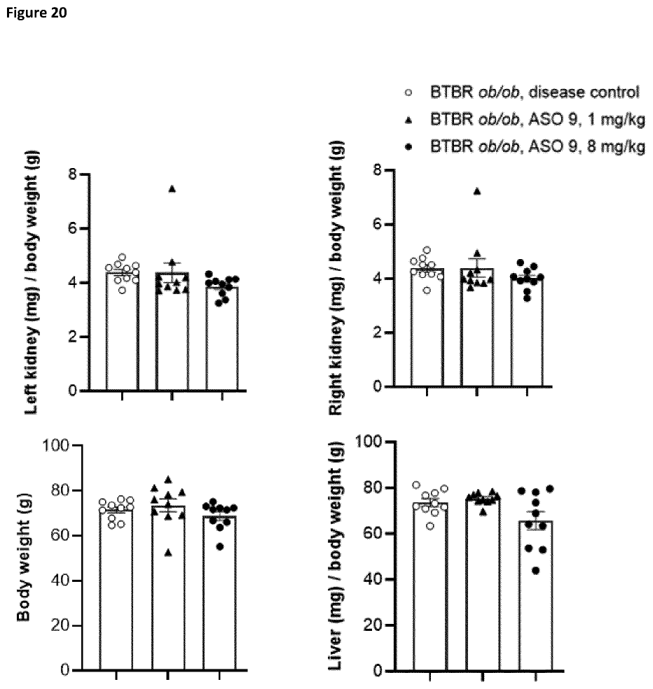

No differences in AST and ALT levels were observed (FIG. 19) and no changes in body or organ weights between ASO 9 treated mice and disease controls (see FIG. 20), results that when taken together indicate that ASO 9 did not have a negative effect on liver enzymes or health status effecting body weight. These results highlight the safety of ARHGEF6 targeting ASOs supporting the suitability of targeting ARHGEF6 in therapy.

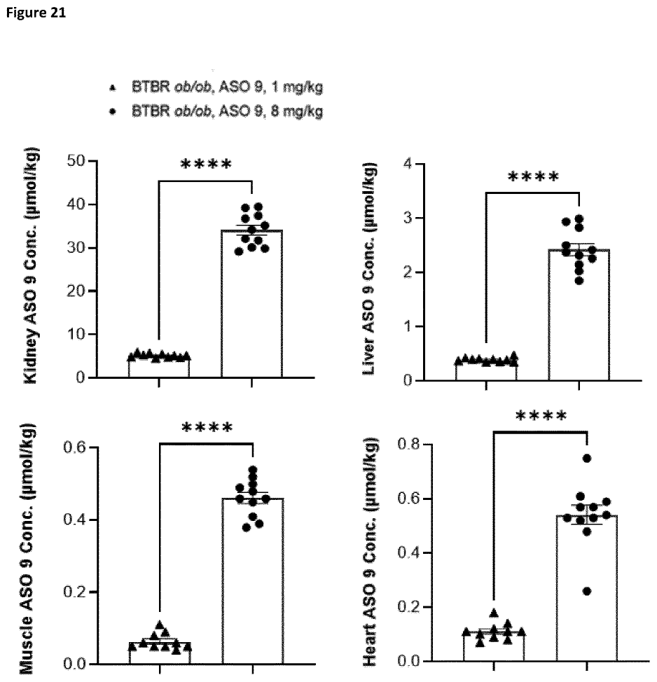

As can be seen in FIG. 21, the quantification levels of ASO 9 in different organs, confirms that the ASO exposure is by far elevated in the kidney, more than 14 times higher, compared to exposure levels in liver, muscle or heart. ASO accumulation following IP dosing is therefore observed in the target tissue (kidney).

Taken together, the results in the BTBR ob/ob model deliver proof of concept that reducing ARHGEF6 protein, as achieved in this study by administration of the ARHGEF6 ASO 9 to BTBR ob/ob mice, can improve kidney function by restoring the glomerular filtration barrier in the kidney. This is shown by reduced loss of proteins (UACR) via the renal filtration barrier as a result of the improved health of the cells that make up the filtration barrier, the podocytes and the endothelial cells. This is also proven by the restoration of kidney glomerular pathology (glomerular score). In summary, the positive results obtained in this disease relevant model supports the proposition that ASOs selectively targeting ARHGEF6 have the potential to restore renal function in CKD involving glomerular filtration barrier dysfunction. These results also support the therapeutic potential of other modalities that deliver a reduction in ARHGEF6 at the protein level such as PROTACS or small molecules that cause degradation of ARHGEF6 protein.

ASO architectures other than the 3-10-3 LNA-DNA-LNA gapmers described above could equally be applied for the generation of ASOs to ARHGEF6. For example, LNA units can be replaced with alternative bridged nucleic acids such as (S)-cEt or ENA (see Morita et al, Bio Med Chem Lett 2002, 12(1), p73-76) and/or the length of the three component units of the gapmer can be adjusted.

Gapmers featuring alternative substitution at the 2′-position of some, or all, ribose units such as 2-OH (RNA), 2′-O-methyl (2′-OMe), 2′-O-methoxyethyl (2′-O-MOE) and 2′-fluoro (2′-F RNA) can also be used to tailor ASO properties (see Deleavey and Damha, Chem Biol, 2012, 19(8), p937-54 for a review on the design of ASOs and Shen and Corey, Nucleic Acids Research, 2018, 46(4), p1584-1600).

Replacement of ribose units in the component ribonucleic acid monomers with morpholino (PMO) units is also possible. Finally, some, or all, of the phosphate linkers between 3′-O and 5′-O groups of adjacent ribonucleic acid monomer units (that provides an overall phosphodiester unit in the oligomer) in the oligonucleotide can be replaced with phosphorothioate (PS), thio-phosphoramidate (NP) or boranophosphate linkages.

In embodiments, there is provided a ARHGEF6 selective antisense oligonucleotide having a gapmer structure

$${( A )}_{0 - 6} - {( DNA )}_{8 - 14} - {( C )}_{0 - 6}$$

in which:
(A)₀₋₆ and (C)₀₋₆ independently denote units comprising from 0 to 6 modified ribonucleic acid monomers (as denoted by the subscript numerals) and
(DNA)₈₋₁₄ denotes a unit comprising from 8 to 14 2-deoxyribonucleic acid monomers nucleotides. As the skilled person will understand, the ribonucleic acid monomer units from which the A and C motifs are composed are covalently linked through phosphodiester bonds between the 3′ and 5′ carbons of adjacent monomer ribose units. DNA units are similarly linked, albeit the phosphodiester bonds are between the 3′ and 5′ carbons of adjacent monomer 2-deoxyribose units

In these gapmer ASO structures the modified ribonucleic acid monomers from which A and C are comprised are independently selected from LNA, (S)-cEt, RNA, 2′-OMe, 2′-O-MOE or 2′-F nucleotide motifs or a nucleoside in which the ribose is replaced with a morpholino (PMO) group. In these gapmers, some or all of the phosphodiester linkages may be replaced by phosphorothioate (PS) or thiophosphoramidate groups or thio-phosphoramidate (NP) or boranophosphate linkage.

Examples of chemical modifications of internucleotide linkages that may be used in ARHGEF6 ASOs are presented below (taken from Chem Biol, 2012, 19(8), p937-54).

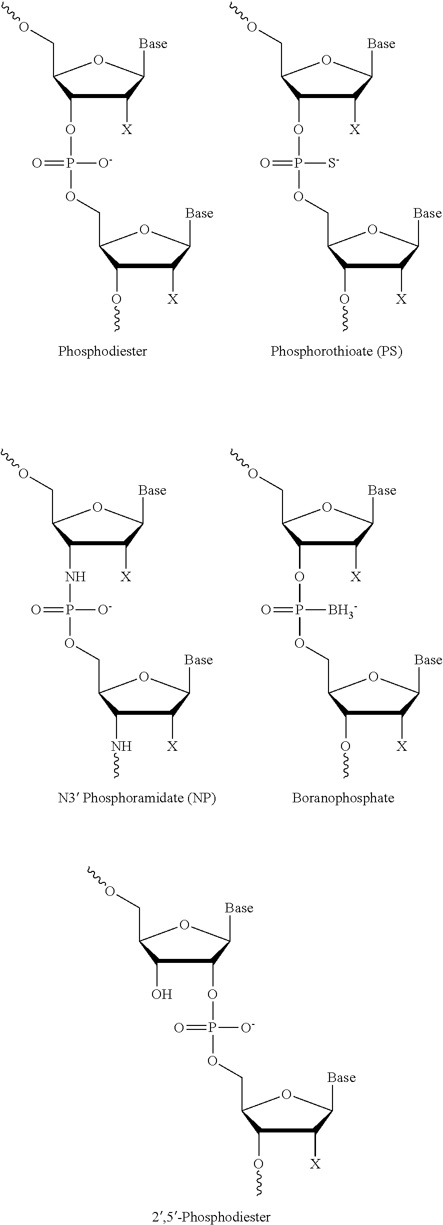

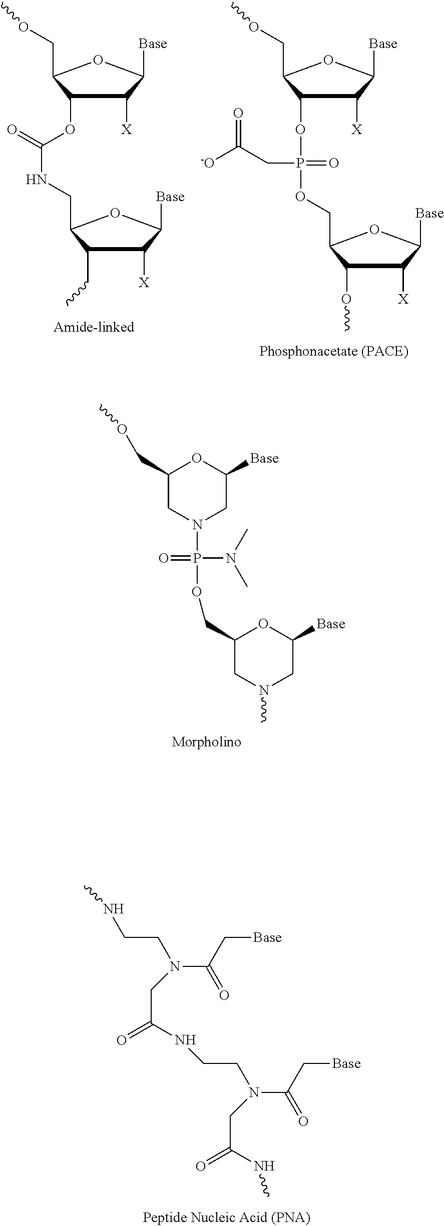

Examples of chemical modifications of oligonucleotide sugars that may be used in ARHGEF6 ASOs are presented below (taken from Chem Biol, 2012, 19(8), p937-54).

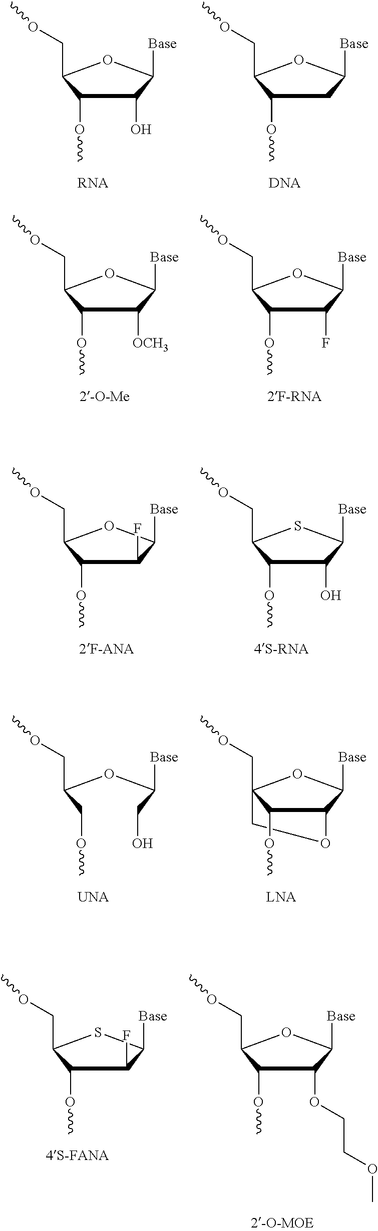

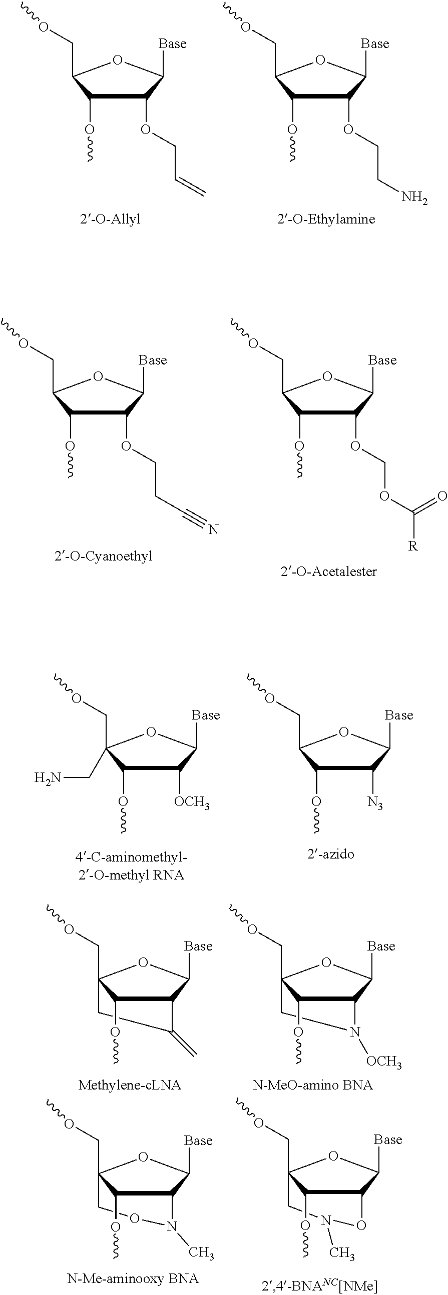

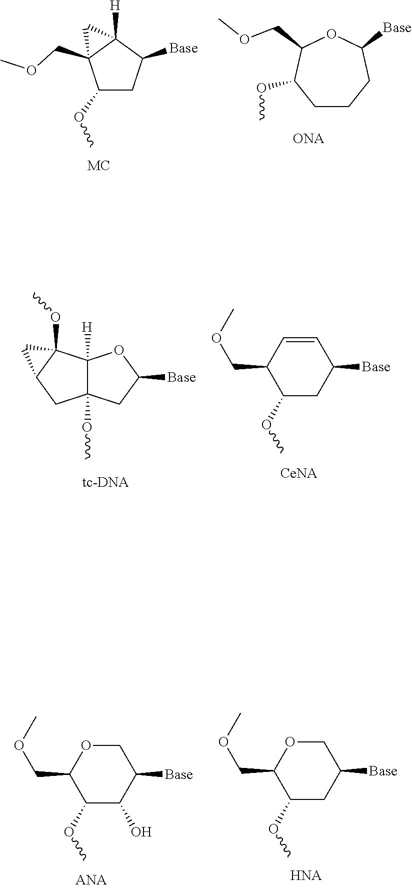

In parallel with the development of inhibitors of ARHGEF6 activity, we sought to further understand the interactions of ARHGEF6 with its protein partners.

ARHGEF6 is a scaffolding protein and drives its biological function through interactions with key protein partners. To investigate the role of ARHGEF6 in the activation of Rac1 we interrogated the interaction of ARHGEF6 with PAK1 and GIT1. PAK1 is a member of the p21-activated kinase family that has been implicated in regulating cell motility and morphology. GIT1, G-protein-coupled receptor (GPCR)-kinase-interacting proteins 1, is a ubiquitous multidomain GTPase activating protein and is proposed to act as a scaffolding protein to promote multiprotein interactions in diverse cellular processes.

A peptide from PAK1 (amino-acid sequence: DDDATPPPVIAPRPEHTKSVYTR (SEQ ID NO: 17)) was found to bind to full-length ARHGEF6 (residues 1-776) with a potency of 13.6 M in a surface plasmon resonance (SPR) experiment. Further exploration of the interaction was carried out with a shortened ARHGEF6 construct (SH3-DH-PH construct=amino-acid residues 155-551) for which the peptide of PAK1 displayed specific binding with a potency of 7.3 M and 9.6 M applying a steady-state model and a 1:1 kinetic interaction model respectively. The binding of the peptide to this construct was further confirmed by using isothermal titration calorimetry (ITC), yielding a binding affinity of 4.3 μM.

Interaction with PAK1 and GIT1 were tested for hARHGEF6 isoform 2 (M155-P776). In SPR experiments, PAK1 was observed to transiently interact with ARHGEF6. The association (kₒₙ) and dissociation (k_{off}) rate constants were estimated to be 7.6×10⁵ M⁻¹s⁻¹ and 1.6×10⁻² s⁻¹, respectively, resulting in an apparent dissociation constant (K_{D}) of 21.1 nM.

GIT1, on the other hand, exhibited slow association but a very stable complex formation with ARHGEF6. Due to the slow kinetics, particularly the dissociation phase, the values of the rate constants could not be uniquely determined from SPR curves. However, analysis of the binding suggests the kₒₙ and k_{off} values to be ~1.7×10⁴ and <10⁵, respectively.

Based on the estimation of Rₘₐₓ values, a monomer of PAK1 was bound per 13.7 monomers of immobilized ARHGEF6 on the SPR chip. It is likely that ARHGEF6 exists in heterogenous states (potentially different conformational states or post translational modifications) and only a sub-set of ARHGEF6 protomers are capable of binding to PAK1. Similar analysis with GIT1 suggests a binding ratio of 1:2.4 (GIT1:ARHGEF6).

As noted above the present specification provides methods for the treatment or prophylaxis of chronic disease, inhibitors of ARHGEF6 activity for use in the treatment of chronic kidney disease and inhibitors of ARHGEF6 activity for use in the manufacture of a medicine.

Accordingly, the present specification provides a method for the treatment or prophylaxis of chronic kidney disease comprising administering an inhibitor of ARHGEF6 activity to a patient in need thereof.

In certain examples, the inhibitor of ARHGEF6 activity for use in the method of treatment of CKD lowers ARHGEF6 expression at the protein level. Examples of inhibitors of ARHGEF6 activity include antisense oligonucleotides to ARHGEF6 mRNA that cause downregulation of ARHGEF6 or ARHGEF6 proteolysis targeting chimeras (PROTACs) or small molecules that cause degradation of ARHGEF6 protein. In certain examples, the inhibitor of ARHGEF6 activity acts in a catalytic manner.

In embodiments, the method of treatments and uses of the specification are for CKD, for example diabetic nephropathy (DN), focal segmental glomerulosclerosis (FSGS), renal hypertension (HTN), IgA nephropathy/Berger's disease (IgAN), rapidly progressive glomerulonephritis (RPGN) and systemic lupus erythematosus (SLE).

In embodiments, the method of treatments and uses of the specification are for CKD involving glomerular filtration barrier dysfunction.

The inhibitor of ARHGEF6 activity may act by reducing expression of ARHGEF6 protein, as is the case with an antisense oligonucleotide (ASO) inhibitor that binds to the mRNA produced by ARHGEF6, or may directly cause degradation of ARHGEF6 on a protein level, as is the case with a proteolysis targeting chimera (PROTAC, see e.g. Sun et al, Signal Transduction and Targeted Therapy volume 4, Article number: 64 (2019)) or small molecule degraders.

In embodiments of the specification, there are provided methods for treating chronic kidney disease comprising administration of an antisense oligonucleotide to ARHGEF6 mRNA to a patient in need thereof, thereby causing depletion of ARGHEF6 protein. In certain embodiments the ASO is a RNase H competent ASO, i.e. an ASO that binds to the cognate mRNA transcript to form a RNA-DNA heteroduplex that is recognised by the endogenous RNase H enzyme RNASEH1 such that the RNA is catalytically degraded by the enzyme thereby releasing the ASO to bind with further mRNA, thereby silencing ARHGEF6 expression and reducing ARHGEF6 protein levels.

In embodiments, the patient in need of treatment with an inhibitor of ARHGEF6 activity is a patient identified as having Stage 2 or Stage 3 CKD. In embodiments, the patient in need of treatment with an inhibitor of ARHGEF6 activity is a patient identified as having Stage 4 or Stage 5 CKD. In embodiments the patient in need of treatment is identified on the basis of their Stage of CKD as established by their eGFR as measured before the onset of treatment. In embodiments, the patient in need of treatment is identified on the basis of their Stage of CKD as established by their eGFR as measured before the onset of treatment, for example a measured eGFR of <90 mL/min/1.73 m² or <70 mL/min/1.73 m² or <60 mL/min/1.73 m². In embodiments, the patient in need of treatment is identified on the basis of their having GFR of <60 mL/min/1.73 m². In embodiments, the patient in need of treatment is identified on the basis of their Stage of CKD as established by their Urine Albumin-to-Creatinine Ratio (UACR) as measured before the onset of treatment, for example the patient has a UACR of >30 mg/g, for example a UACR of 200 to 5000 mg/g. Methods for diagnosing chronic kidney disease and CKD Stage are well known in the art. A comprehensive overview of the techniques used to evaluate CKD are provided in the 2024 KDIGO Guidelines at Chapter 1 (pages S169-S195).

In embodiments herein wherein GFR or eGFR values are specified they may, for example, be established on the basis of analysis of serum creatinine levels (see e.g. Cockcroft, Nephron 1976:16:31-41). Alternatively, the eGFR value may be calculated on the basis of a composite of serum creatinine and cystatin C level, for example in combination with data on the patient's age, race and gender as per the 4 variable MDRD study equations (see e.g. Inker et al, N Eng J Med 2012 Jul 5; 367(1):20-9; www.mdrdcom). Other methods for measuring GFR or EGFR will be apparent to the skilled reader.

In embodiments of the specification, there is provided a method for treating chronic kidney disease comprising administration of a proteolysis targeting chimera (PROTAC) that causes degradation of ARHGEF6 to a patient in need thereof (see Paiva and Crews, Curr Opin Chem Biol 2019, 50, 111-119 for a review of PROTACs). The protein degradation event, mediated by a E3 ubiquitin ligase, releases the PROTAC allowing it to function in a catalytic manner.

In embodiments of the specification, there is provided a method for treating chronic kidney disease comprising administration of a degrader of ARHGEF6, for example a small molecule or a PROTAC, to a patient in need thereof.

In embodiments of the specification, there is provided a method for treating chronic kidney disease comprising administration of an inhibitor of ARHGEF6 activity to a patient in need thereof. In embodiments, the inhibitor may prevent, reverse or reduce the ARHGEF6 mediated activation of Rac1 and/or the ARHGEF6 mediated depletion of active (β1-integrins levels relative to that observed prior to treatment with the ARHGEF6 inhibitor. In embodiments the inhibitor of ARHGEF6 activity is capable of preventing or reducing the extent of the ARHGEF6 mediated activation of Rac1 and preventing or reducing the extent of the ARHGEF6 mediated depletion of active (β1-integrins levels relative to that observed prior to treatment with the ARHGEF6 inhibitor.

In embodiments of the specification the inhibitor of ARHGEF6 activity is an agent that binds to ARHGEF6 mRNA, for example a ARHGEF6 ASO, thereby triggering degradation of ARHGEF6 mRNA and, consequently, a decrease in intracellular concentrations of ARHGEF6 protein. In embodiments the inhibitor of ARHGEF6 activity is an agent that binds to ARHGEF6 protein that elicits the degradation of ARHGEF6 and, consequently, triggers a decrease in intracellular concentrations of ARHGEF6 protein.

In embodiments the inhibitor of ARHGEF6 activity binds directly to ARHGEF6, thereby inhibiting the ARHGEF6 mediated activation of Rac1 and/or the ARHGEF6 mediated depletion of active (β1-integrin levels. In embodiments the inhibitor of ARHGEF6 activity binds directly to ARHGEF6, thereby inhibiting the ARHGEF6 mediated activation of Rac1. In embodiments the inhibitor of ARHGEF6 activity binds directly to ARHGEF6, thereby inhibiting the ARHGEF6 mediated depletion of active (β1-integrin levels.

In such embodiments the inhibitor of ARHGEF6 activity may bind to ARHGEF6 in a reversible or irreversible manner.

In embodiments, the inhibitor of ARHGEF6 activity may reduce, arrest or reverse the rate of progression of chronic kidney disease, for example as assessed by the reduction in GFR or eGFR or increase in UACR. The assessment of rate of progression of chronic kidney disease may be assessed by comparing the rate of change of the measured GFR, eGFR or UACR in chronic kidney disease patients treated with an inhibitor of ARHGEF6 activity versus that in chronic kidney disease patients with comparable measured GFR, eGFR or UACR that are treated with placebo or an alternative treatment for chronic kidney disease.

In embodiments, the inhibitor of ARHGEF6 is for use in the treatment of patients on the cusp of developing Stage 3 CKD or with Stage 3 CKD, i.e. patients with a GFR of <70 mL/min/1.73 m² or a GFR between 30 to 59 mL/min/1.73 m². In some embodiments the inhibitor of ARHGEF6 is for use in the treatment of a patient with Stage 4 or Stage 5 CKD, i.e. a patient with a GFR<30 mL/min/1.73 m².

In embodiments, the method of treatment of CKD further comprises identifying a patient for treatment on the basis of the patient having a GFR, eGFR or UAR indicating that the patient has Stage 2, Stage 3 or Stage 4 CKD.

In embodiments, the method of treatment further comprises selecting the patient in need thereof for treatment with an inhibitor of ARHGEF6 activity, wherein the inhibitor of ARHGEF6 activity is an agent capable of inhibiting the ARHGEF6 mediated activation of Rac1 or the ARHGEF6 mediated reduction in levels of activated β-integrin or an agent capable of reducing the expression of ARHGEF6 or an agent capable of degrading ARHGEF6 at a protein level thereby reducing intracellular ARHGEF6 concentrations.

In an aspect of the specification there is provided an inhibitor of ARHGEF6 activity for use in the treatment or prophylaxis of chronic kidney disease. In embodiments of the specification the inhibitor of ARHGEF6 activity for use is capable of reducing the amount of ARHGEF6 at a protein level, thereby reducing the net ARHGEF6 activity. In embodiments of the specification the inhibitor of ARHGEF6 activity is an agent that can reduce the ARHGEF6 mediated activation of Rac1 and/or the ARHGEF6 mediated depletion of active β1-integrins levels.

The inhibitor of ARHGEF6 activity for use may act by reducing expression of ARHGEF6 protein, as is the case with an anti-sense oligonucleotide (ASO) inhibitor that binds to the mRNA produced by ARHGEF6, or can directly cause degradation of ARHGEF6 on a protein level, as is the case with a proteolysis targeting chimera (PROTAC) or a small molecule ARHGEF6 degrader.

In a further aspect of the specification there is provided an antisense oligonucleotide (ASO) to ARHGEF6 mRNA for use in the treatment of CKD. In a further aspect of the specification there is provided an antisense oligonucleotide (ASO) to human ARHGEF6 mRNA for use in the treatment of CKD in a human patient in need thereof.

In embodiments the inhibitor of ARHGEF6 activity for use is for use in a patient identified as having Stage 2 or Stage 3 CKD. In embodiments the inhibitor of ARHGEF6 activity for use is for use in a patient identified as having Stage 4 or Stage 5 CKD. In embodiments the inhibitor of ARHGEF6 activity for use is for use in a patient identified on the basis of their Stage of CKD as established by their eGFR as measured before the onset of treatment. In embodiments the inhibitor of ARHGEF6 activity for use is for use in a patient identified on the basis of their Stage of CKD as established by their eGFR as measured before the onset of treatment, for example a measured eGFR of <90 mL/min/1.73 m² or <70 mL/min/1.73 m². In embodiments the inhibitor of ARHGEF6 activity for use is for use in a patient identified on the basis of their Stage of CKD as established by their Urine Albumin-to-Creatinine Ratio (UACR) as measured before the onset of treatment, for example a patient that has a UACR of >30 mg/g, for example a UACR of 200 to 5000 mg/g.

In a further aspect of the specification there is provided an ARHGEF6 PROTAC for use in the treatment of CKD.

In embodiments, the use of the inhibitor of ARHGEF6 activity for the treatment of chronic kidney disease comprises administration of a proteolysis targeting chimera (PROTAC) that causes degradation of ARHGEF6.

In embodiments, the use of the inhibitor of ARHGEF6 activity for the treatment of chronic kidney disease comprises administration of a degrader of ARHGEF6, for example a small molecule or a PROTAC.

In embodiments, the inhibitor of ARHGEF6 activity for use in the treatment of chronic kidney disease is administered orally. In embodiments, the inhibitor of ARHGEF6 activity for use in the treatment of chronic kidney disease is administered intravenously (IV). In embodiments the inhibitor of ARHGEF6 activity is capable of preventing the ARHGEF6 mediated activation of Rac1 and/or the ARHGEF6 mediated depletion of active β1-integrins levels. In embodiments the inhibitor of ARHGEF6 activity is capable of preventing the ARHGEF6 mediated activation of Rac1 and the ARHGEF6 mediated depletion of active (β1-integrins levels.

In embodiments of the specification the inhibitor of ARHGEF6 activity for use in the treatment of chronic kidney disease is an agent that binds to ARHGEF6 mRNA thereby triggering degradation of ARHGEF6 mRNA and, consequently, a decrease in intracellular concentrations of ARHGEF6 protein. In embodiment the inhibitor of ARHGEF6 activity for use in the treatment of chronic kidney disease is an agent that binds to ARHGEF6 and in so doing elicits the degradation of ARHGEF6 and, consequently, a decrease in intracellular concentrations of ARHGEF6 protein.

In embodiments, the inhibitor of ARHGEF6 activity binds directly to ARHGEF6 thereby inhibiting the ARHGEF6 mediated activation of Rac1 and/or the ARHGEF6 mediated depletion of active (β1-integrin levels. In embodiments, the inhibitor of ARHGEF6 activity binds directly to ARHGEF6 thereby inhibiting the ARHGEF6 mediated activation of Rac1. In embodiments, the inhibitor of ARHGEF6 activity may bind directly to ARHGEF6 thereby inhibiting the ARHGEF6 mediated depletion of active (β1-integrin levels.

In embodiments, the inhibitor of ARHGEF6 activity binds directly to a protein that associates with ARHGEF6 in order to activate Rac1, thereby inhibiting the ARHGEF6 mediated activation of Rac1. In embodiments, the inhibitor of ARHGEF6 activity binds to a complex of ARHGEF6 and at least one other protein, thereby inhibiting the ARHGEF6 mediated activation of Rac1. In embodiments, the inhibitor of ARHGEF6 activity binds to a complex of ARHGEF6 and at least one other protein thereby inhibiting the ARHGEF6 mediated depletion of active (β1-integrin levels.

In embodiments, the inhibitor of ARHGEF6 activity for use reduces, arrests or reverses the rate of progression of chronic kidney disease, for example as assessed by the reduction in GFR or eGFR or increase in UACR. The assessment of rate of progression of chronic kidney disease may be assessed by comparing the rate of change of the measured GFR, eGFR or UACR in chronic kidney disease patients treated with an inhibitor of ARHGEF6 activity versus that in chronic kidney disease patients treated with placebo or an alternative treatment for chronic kidney disease.

In embodiments, the inhibitor of ARHGEF6 is for use in the treatment of Stage 3 CKD, i.e. in a patient with a GFR of <70 mL/min/1.73 m² between 30 to 59 mL/min/1.73 m². In embodiments the inhibitor of ARHGEF6 is for use in the treatment of Stage 4 or Stage 5 CKD, i.e. in a patient with a GFR below 30 mL/min/1.73 m².

In embodiments, the inhibitor of ARHGEF6 is for use in the treatment of CKD in a patient identified for treatment on the basis of having a GFR, eGFR or UAR indicative of Stage 2, Stage 3 or Stage 4 CKD.

In embodiments, the inhibitor of ARHGEF6 is for use in the treatment of CKD is an agent capable of inhibiting the ARHGEF6 mediated activation of Rac1 or the ARHGEF6 mediated reduction in levels of activated β-integrin or an agent capable of reducing the expression of ARHGEF6 or an agent capable of degrading ARHGEF6 at a protein level thereby reducing intracellular ARHGEF6 concentrations.

In an aspect there is provided an ARHGEF6 ASO. In certain embodiments the ARHGEF6 ASO of is selective for ARHGEF6 mRNA, i.e. it has perfect complementary only to ARHGEF6 mRNA, for example human ARHGEF6.

In embodiments the ARHGEF6 ASO a) targets a region of ARHGEF6 having an accessibility score>0.001 (using the Vienna RNA RNAplfold algorithm); b) has perfect complementarity to only the ARHGEF6 sequence; c) has complementarity with 1 mismatch to no greater than 50 other genes; d) does not target a region with a minor allele frequency>0.05, e) has no CG motifs; and f) has a % GC content of >10.

In embodiments, there is provided a ARHGEF6 ASO having a gapmer structure

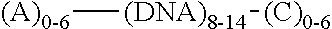

in which:
(A)₀₋₆ and (C)₀₋₆ independently denote units comprising from 0 to 6 modified ribonucleic acid monomers;
(DNA)₈₋₁₄ denotes a unit comprising from 8 to 14 2-deoxyribonucleic acid monomers; and
wherein the subscript denotes the number of monomers from which A, B and C are constituted.

In embodiments, the ARHGEF6 ASO is a 3-10-3 LNA-DNA-LNA gapmer.

In one aspect there is provided an ARHGEF6 ASO produced by a process comprising the steps of a) selecting a ASO that targets an accessible region of ARHGEF6, optionally a region of ARHGEF6 having an accessibility score>0.001 as determined using the Vienna RNA RNAplfold algorithm; b) determining that the candidate ASO has perfect complementarity to only the ARHGEF6 sequence; c) determining that the candidate ASO has complementarity with 1 mismatch to no greater than 50 other genes; d) filtering to ensure that the candidate ASO does not target a region with a minor allele frequency of >0.05, e) ensuring that the candidate ASO has no CG motifs and has a % GC content of >10, and, optionally, f) synthesising the resultant candidate oligonucleotide.

In embodiments, the resultant ARHGEF6 ASO product of the process has a gapmer structure

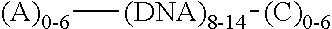

in which:
A and C independently denote units comprising from 0 to 6 modified ribonucleic acid monomers; DNA denotes a unit comprising 2-deoxyribonucleic acid monomers; and
wherein the respective subscripts denote the number of monomers from which A, B and C are constituted.

In embodiments, the resultant ARHGEF6 ASO product of the process is a 3-10-3 LNA-DNA-LNA gapmer.

In one aspect there is provided a ARHGEF6 ASO for use in the treatment of chronic kidney disease. In embodiments, the ARHGEF6 ASO for use in the treatment of chronic kidney disease has a gapmer structure

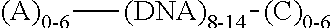

in which:
A and C independently denote units comprising from 0 to 6 modified ribonucleic acid monomers;
DNA denotes a unit comprising 2-deoxyribonucleic acid monomers; and
wherein the subscript denotes the number of monomers from which A, B and C are constituted In embodiments, the ARHGEF6 ASO for use in the treatment of chronic kidney is a 3-10-3 LNA-DNA-LNA gapmer.

In one aspect there is provided method of treatment for chronic kidney disease comprising administering a ARHGEF6 ASO to a patient in need thereof. In embodiments, the ARHGEF6 ASO for use in the method of treatment has a gapmer structure

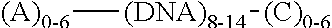

in which:
A and C independently denote units comprising from 0 to 6 modified ribonucleic acid monomers;
DNA denotes a unit comprising 2-deoxyribonucleic acid monomers; and
wherein the subscript denotes the number of monomers from which A, B and C are constituted in which:
A and C independently denote units comprising from 0 to 6 modified ribonucleic acid monomers (as denoted by the subscript numerals); and
DNA denotes a unit comprising from 8 to 14 2-deoxyribonucleic acid monomers.

In embodiments, the ARHGEF6 ASO for use in the method of treatment of chronic kidney is a 3-10-3 LNA-DNA-LNA gapmer.

In an aspect there is provided an ARHGEF6 PROTAC.

In an aspect there is provided an inhibitor of ARHGEF6 activity for use in the manufacture of a medicament, for example a medicament for intended for the treatment of chronic kidney disease.

In embodiments the inhibitor of ARHGEF6 activity for use in the manufacture of a medicament is an ARHGEF6 ASO having a gapmer structure

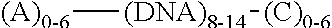

in which:
A and C independently denote units comprising from 0 to 6 modified ribonucleic acid monomers; DNA denotes a unit comprising 2-deoxyribonucleic acid monomers; and
wherein the respective subscripts denote the number of monomers from which A, B and C are constituted. In embodiments, the ARHGEF6 ASO for use in the manufacture of a medicament has a 3-10-3 LNA-DNA-LNA gapmer structure.

In embodiments the medicament comprising an ARHGEF6 ASO is for use in the treatment of chronic kidney disease. In embodiments the medicament comprising an ARHGEF6 ASO is for use in the treatment of chronic kidney disease involving glomerular filtration barrier dysfunction.

In embodiments, the medicament for the treatment of CKD is intended for use in a patient identified for treatment on the basis of having a GFR, eGFR or UACR indicative of Stage 2, Stage 3 or Stage 4 CKD.

In embodiments the medicament is for use in the treatment of Stage 3 CKD, i.e. in a patient with a GFR of <70 mL/min/1.73 m² between 30 to 59 mL/min/1.73 m². In embodiments the inhibitor of ARHGEF6 is for use in the treatment of Stage 4 or Stage 5 CKD, i.e. in a patient with a GFR below 30 mL/min/1.73 m².

In an aspect there is provided a pharmaceutical composition comprising an inhibitor of ARHGEF6 activity. In embodiments the pharmaceutical composition is for oral administration. In embodiments the pharmaceutical composition is for IV administration. In embodiments the pharmaceutical composition is for intramuscular administration.

In an aspect there is provided a kit containing a pharmaceutical composition comprising an inhibitor of ARHGEF6 activity and instructions for its use in the treatment of CKD.

In a further aspect of the specification there is provided a method of identifying a patient for treatment with an inhibitor of ARHGEF6 activity comprising the steps of analysing a sample obtained from a patient for the presence of a biomarker of chronic kidney disease, for example a direct or surrogate marker of elevated levels of ARHGEF6, Rac1 activation or (β1-integrin inactivation or for a marker of CKD, such as measured GFR or eGFR or UACR.

In one embodiment the specification provides a method of treatment or prophylaxis of CKD comprising administering an ARHGEF6 inhibitor to a patient in need thereof. The patient may be identified as having impaired kidney function based on their eGFR value. The eGFR value may, for example, be established on the basis of analysis of serum creatinine levels (see e.g. Cockcroft, Nephron 1976:16:31-41). Alternatively, the eGFR value may be calculated on the basis of a composite of serum creatinine and cystatin C level, for example in combination with data on the patient's age, race and gender as per the 4 variable MDRD study equations (see e.g. Inker et al, N Eng J Med 2012 Jul 5; 367(1):20-9; wwwmdrdcom).

In embodiments, the patient in need of treatment with an ARHGEF6 inhibitor may be identified as having CKD on the basis of a measured glomerular filtration rate (GFR). For example, a measured GFR of <60 ml/min/1.73 m² is indicative of stage 3, or more advanced, CKD (see KDIGO) and may be used to identify a patient as suitable for treatment with a ARHGEF6 inhibitor. The patient for treatment may also be identified as having early stage CKD, with intervention with an ARHGEF6 inhibitor being used to halt, delay or substantially reduce, disease progression.

The identification of a patient in need of treatment with an ARHGEF6 inhibitor may also be established by an existing test or a composite of other existing tests known for detecting impaired kidney function. For example, measurement of the amount of albumin in a patient's urine (an albumin-to-creatinine (ACR) test), with an ACR of above 30 mg/g can be indicative of kidney disease. eGFR may equally be used to identify and classify the extent of impaired kidney function and the need for therapeutic intervention with a ARHGEF6 inhibitor.

A patient in need of treatment with an ARHGEF6 inhibitor may also be identified by obtaining a renal biopsy sample from the patient and analysing whether the sample exhibits elevated levels of ARHGEF6. Advantageously, as ARHGEF6 overexpression is seen to initiate at CKD stage 1, the use of an ARHGEF6 inhibitor may be indicated at, and initiated at, an earlier stage of CKD than would otherwise be possible. Intervention with an ARHGEF6 inhibitor may be used to reverse, arrest or slow the decline in a patient's kidney function.

In one embodiment, the specification provides a method of treatment or prophylaxis of a patient diagnosed as having, or at risk of developing, CKD comprising administering to the patient a therapeutically effective dose of an ARHGEF6 inhibitor. The patient may, for instance, be diagnosed has having 1, 2 or 3 CKD on the basis of, for example, their urine Albumin-to-Creatinine ratio or their urine Protein-to-Creatinine ratio, their measured GFR or their eGFR. The patient may, for instance, be diagnosed has having 1, 2 or 3 CKD on the basis of any of the techniques recommended in the 2024 KDIGO Guidelines.

In one embodiment, the specification provides a method of reducing the risk of CKD in a patient who is suspected to have a propensity for developing CKD or for progression to a later stage of CKD, for example a patient identified as overexpressing ARHGEF6 or ARHGEF6 from the results of renal biopsy or on the basis of their urine Albumin-to-Creatinine ratio, their urine Protein-to-Creatinine ratio, their measured GFR or their eGFR, comprising administering to the patient a therapeutically effective amount of an ARHGEF6 inhibitor.

In one embodiment, the specification provides a method of reducing the risk of CKD in a patient who is suspected to have a propensity for developing CKD, comprising administering to the patient a therapeutically effective amount of an ARHGEF6 inhibitor.

In one embodiment, the specification provides a method of treating or preventing CKD in a subject comprising the step of administering an ARHGEF6 inhibitor to the subject, to thereby treat or prevent CKD in the subject. Preventing CKD in such embodiments can refer to the act of arresting, reversing or reducing the rate of reduction in the eGFR or measured GFR such that stage 3 CKD does not progress to stage 4 CKD or that a patient identified as having a certain stage of CKD at the outset of treatment sees an improvement in their measured or estimated GFR after a period of time on treatment with an ARHGEF6 inhibitor.

In one embodiment, the specification provides a method of treating a subject with CKD, the method comprising administering an ARHGEF6 inhibitor to a patient in need thereof, wherein the method of treatment reverses or arrests the reduction of, or reduces the rate of reduction of, the patients eGFR.

In one embodiment, the specification provides a method of monitoring the response of a patient having CKD with an ARHGEF6 inhibitor, the method comprising:
(a) assessing the patient's eGFR or GFR following treatment with an ARHGEF6 inhibitor, and
(b) comparing this eGFR or GFR with the eGFR or GFR level measured prior to commencement of treatment with the ARHGEF6 inhibitor.

In such an embodiment the determination of the patient's eGFR or GFR following administration of the ARHGEF6 inhibitor can be after treatment of with an ARHGEF6 inhibitor for a period of one week, two weeks, one month or more, for example, 6 months or one year.

In examples according to the specification the ARHGEF6 inhibitor is administered orally. In examples according to the specification the ARHGEF6 inhibitor is administered by injection, for example into the blood stream or directly to the kidney.

### EXAMPLES

So that the specification may be readily appreciated, reference is made to the examples below.

### Cell Culture

Inducible CAS9 cell line HEK-ODIN was generated at AstraZeneca R&D Gothenburg (see US2018305714 (A1) for detail). HEK-ODIN cells were cultured in Dulbecco's Modified Eagle Medium (DMEM, Gibco™, Thermo Fisher Scientific) supplemented with 10% Fetal Bovine Serum (FBS, Gibco™, Thermo Fisher Scientific) and 1% Penicillin-Streptomycin (Pen/Strep, Thermo Fisher Scientific) in a 37° C. and 5% CO₂ incubator. CAS9 expression was induced with doxycycline 1 μg/mL overnight treatment. Bristol human podocyte line (obtained from Prof. Saleem, Bristol University) was maintained and expanded in RPMI-1640 medium (Merck) supplemented with Insulin-Transferrin-Selenium, 10% FBS, and 1% Pen/Strep in a 33° C. and 5% CO₂ incubator. Before the experiment, the Bristol human podocyte cell line was seeded 25,000 cells/cm² and transferred to 37° C. culture for 14 days. Podocytes were stimulated with puromycin aminonucleoside (PAN, Sigma-Aldrich) 100 or 1000 ng/μl for 72 h; palmitic acid (Sigma-Aldrich) 100 or 300 μM conjugated with human serum albumin (HSA, Sigma-Aldrich) for 24 h; protamine sulfate (Sigma-Aldrich) 600 μg/mL for 30 or 60 min.

Construction of the ARHGEF6 sgRNA Expressing Plasmids and Transfection

To generate efficient ARHGEF6 knockout, a pair of sgRNAs (CRISPR1: AATCAAGGTGCATCGAGCCC; CRISPR2: CAGCAAACCATTCATGCGAC) from previously designed libraries based on RefSeq sequence NM_004840.3 were selected to induce proximate cuts and precisely delete a 47 bp region of ARHGEF6 CDS. The selected sgRNAs were ordered as oligo-duplexes (Sigma-Aldrich) to clone into Aarl restriction sites of pMlu backbone (a sgRNA expressing backbone containing human U6 promoter, constructed in house). This construct contains a human U6 promoter and SpCas9-sgRNA scaffold. Cloning of the oligo duplexes into this construct (between U6 and the scaffold) results in the final expressing sgRNA plasmid. The sequence of the recombinant plasmids (expressing sgRNAs) was Sanger sequence verified. The sgRNA plasmid was transfected into CAS9-induced HEK-ODIN cells with lipofectamine LTX (Thermo Fisher Scientific) following manufacturer's instruction. Genomic DNA was isolated from transfected cells using Qiagen PureGene kit (Catalog No. 158745, Qiagen) and the knockout efficiency of ARHGEF6 was validated using genotyping primers (Forward: CATGAGTGTCTGGCTCACCA; Reverse: GGACCATACTTGGCACACGA) (Sigma-Aldrich).

### Construction of the ARHGEF6 Over-Expression Plasmids and Transfection

To construct the plasmids for over-expression of ARHGEF6 plasmids, eight gBlocks were designed and ordered from GeneArt (Invitrogen). These gblocks include two containing CDS for wild-type ARHGEF6 splice variants (RefSeq NM_004840.3 and NM_001306177.1) and six containing mutations corresponding to six identified human SNPs (X-135762909-G-A, X-135772876-C-T, X-135789128-A-T, X-135790902-T-G, X-135790924-C-T, and X-135795444-G-A) based on RefSeq NM_004840.3. Each gBlock was consisted of: Forward Primer binding site (MM4), EcoRI, Kozak motif, FLAG-Tag, CDS, GS-linker, Xhol, STOP codon, Xbal and Reverse Primer binding site. The received gBlocks were cloned into pMA backbone (Thermo Fisher Scientific). Constructs were digested using EcoRI and Xhol restriction enzymes (NewEngland Biolabs) and inserts were gel purified and cloned into EcoRI and Xhol sites of pCMV-Cas9-2A-GFP plasmid (GenScript). Finally, the recombinant plasmids were purified using Qiagen Miniprep kit and Sanger sequence verified. The final constructs express the ARHGEF6 CDS variants under the control of CMV promoter. A self-cleaving peptide, T2A, is expressed between ARHGEF6 CDS and GFP. The ARHGEF6 over-expression plasmids were transfected into CAS9-induced HEK-ODIN cells or podocytes with lipofectamine LTX (Thermo Fisher Scientific) following manufacturer's instruction.

### Lentiviral Infection of Podocytes

Lentiviral particles to express ARHGEF6-GFP was purchased from Origene (www.origene.com), to overexpress ARHGEF6 shRNA based on RefSeq NM_004840.3 and Rac1 shRNA based on RefSeq NM_006908.5 were purchased from Sigma. After 7-9 days of culture at 37° C., podocytes were infected with lentivirus at MOI (Multiplicity of infection; MOI=Plaque forming units (Pfu)/number of cells) 1 or 10 for 7-5 days. Podocytes were harvested 14 days after 37° C. culture.

### Protein Extraction and Western Blot

Cells were lyzed with RIPA Buffer (Santa Cruz Biotechnology) supplemented with 1× PhosStop and Complete mini (Roche). Total protein lysates were applied to electrophoresis on Novex 4-20% Tris-glycine mini gels and blotted on PVDF membrane. The membrane was incubated with Rabbit monoclonal anti-ARHGEF6 antibody 1:1000 (Cell Signaling Technology) overnight at 4° C., followed by incubation with HRP anti-rabbit secondary antibody 1 h at room temperature. For protein loading control, the membrane was incubated with HRP conjugated mouse monoclonal anti-GAPDH antibody (Abcam) at room temperature for 1 h. Chemiluminescent signal was detected with SuperSignal West Pico Plus reagents (Thermo Fisher Scientific). The images were taken by Bio-Rad ChemiDoc Imaging System and analyzed with the Image Lab 5.2 software.

### Active Rho GTPase Pull Down Assay

The active Rho GTPases Rac1, Cdc42, and RhoA were determined using Active Rac1/Cdc42 or Rho Pull-Down and Detection Kit (Catalog number 16118 and 16116, respectively, Thermo Fisher Scientific) following manufacturer's instruction. Briefly, protein lysis of HEK-ODIN cells containing 0.5 mg protein were applied on Glutathione Resin with GST-Rhotekin-RBD (for active RhoA) or GST-human Pak1-PBD (for active Rac1/Cdc42) in spin cups. The active GTP-bound GTPases in the lysis were bound to resin and retained in the spin cups. After 3 times' washing, the unbound proteins were discarded and the resin bound active Rho GTPase were eluted using 50 μL reducing sample buffer. Twenty-five μL of eluted active GTPase was submitted to western blotting quantification of protein level using anti-Rac1-, anti-Cdc42-, or anti-RhoA-antibody included in the kit. Equal amount of protein lysis without performing pull down assay were also applied on western blotting to determine the total protein level of Rac1, Cdc42, or RhoA. The ratio between active and total Rho GTPase was used as an index of Rho GTPase activity.

### Fluorescence Staining of Podocytes

Podocytes cultured in 96-well black wall plates were washed with phosphate-buffered saline (PBS, Gibco™, Thermo Fisher Scientific) and fixed with 2% paraformaldehyde (PFA, VWR Chemicals), 4% sucrose (Sigma-Aldrich) in PBS for 5 min. Podocytes were permeabilized with 0.3% triton-X (Sigma-Aldrich) in PBS at 4 degrees for 10 min. Podocytes were blocked with blocking buffer containing 2% FBS, 2% bovine serum albumin (BSA, Sigma-Aldrich), and 0.2% Fish gelatin (Sigma-Aldrich) at room temperature for 30 min. After blocking, podocytes were incubated with mouse anti-active 31 integrin (12G10) antibody (1:300, Abcam) at room temperature for 1 h, anti-mouse secondary antibody for 1 h, and followed by Phalloidin (5 Units/mL, Thermo Fisher Scientific) for 30 min. Fluorescence imaging was performed using high-throughput CV7000 Yokogawa confocal microscopy with a 20× objective lens. The number of podocytes with positive stress fibers were counted manually and intensity of Phalloidin and active 31 integrin staining was measured by Columbus Image Analysis System.

### Immunofluorescence Staining of Kidney Sections

Deparaffinisation and antigen retrieval of mouse kidney paraffin sections were performed with rodent decloaker plus hot rinse (Biocare Medical) in a steam cooker (2100 Retriever) for 45 min. Sections were blocked with 1% BSA, 2.5% horse serum, 0.5% Triton X-100 in PBS for 30 min at RT. Sections were incubated with rabbit polyclonal anti-ARHGEF6 antibody 1:100 (Thermo Fisher Scientific) at 4 degrees overnight. After 3 times' washing with PBS, the secondary antibody Alexa Fluor 594 donkey-anti-rabbit IgG (H+L) (Invitrogen, Thermo Fisher Scientific) 1:500 was applied at RT for 1 h. To reduce autofluorescence, sections were incubated in 0.3% Sudan black B (Sigma-Aldrich) in 70% ethanol solution for 25 min in dark. After staining, sections were mounted using Vectashield with DAPI mounting medium (Vector). Fluorescent images were taken using Zeiss Axio Scan.Z1 scanner with 20× objectives.

### Isolation of Mouse Glomerulus

C57BL/6N mice were anesthetised using isoflurane before chest was opened and perfused with 30 mL Hank's balanced salt solution (HBSS, Gibco™, Thermo Fisher Scientific) through left ventricle followed by 30 mL magnetic bead (Catalog No. 140.04, Dynal, Thermo Fisher Scientific) solution. Kidneys were collected after perfusion and cortex was minced on ice. Minced cortex was mixed with 1 mL collagenase A (Catalog No. 103 586, Roche, 1 mg/mL in HBSS) and incubated at 37° C. for 30 min. The glomeruli suspension was collected after passing minced cortex through 100 μm cell strainer twice. The suspension in 50 mL Falcon tube was centrifuged at 4° C. at max speed for 15 minutes. The pellets were re-suspended in HBSS and glomerulus were purified on a magnetic holder. The final glomeruli pellets were collected and resuspended in 1 mL HBSS. Glomeruli were examined and counted under dissecting microscope. About 250 glomeruli were seeded into each well on 24-well plate and cultured with DMEM with 10% FBS in 37° C. 5% CO₂ incubator.

RNA Extraction from Mouse Glomeruli, cDNA Synthesis, and Quantitative RT-PCR

Total RNA was extracted from cells using RNeasy Mini kit (Qiagen), while for mouse glomeruli, total RNA was extracted using RNeasy 96 kit following manufacturer's instruction (Qiagen). cDNA was synthesized using High-capacity cDNA reverse transcription kit following manufacturer's instruction (Applied Biosystems).

Real-Time qPCR

RT-qPCR was performed using Taqman Gene Expression Master Mix on QuantStudio™ 6 Flex Real-Time PCR device (Applied Biosystems). Human ARHGEF6 and 36B4 and mouse Arhgef6 and Hprt Taqman probes were purchased from Applied Biosystem.

### ASO Synthesis

All starting materials, reagents, and solvents were used as received. Unless otherwise stated, solvents and reagents were obtained from Sigma Aldrich.

Oligonucleotide synthesis: Oligonucleotides were synthesized on a 1 μmol scale on an K&A DNA-RNA Synthesizer H-8 SE (K&A Laborgeraete GbR, 64850 Schaafheim, Germany) using controlled-porosity glass support with universal CUTAG linker (27 μmol/g, purchased from HTI Automation GmbH, 85560 Ebersburg, Germany). All phosphoramidites (Sigma Aldrich) were dissolved to a final concentration to 0.1 M (22 equivalents) in DNA-grade acetonitrile prior to use. Detritylation was performed using 3% dichloroacetic acid in dichloromethane. Activator 42® (5-[3,5-Bis(trifluoromethyl)phenyl]-1H-tetrazole solution, 0.25 M in acetonitrile,) was used as activating agent for the couplings. Coupling times for phosphoramidites were 1 min for DNA building blocks and 7 min for LNA building blocks. Sulfurizing Agent II (3-((Dimethylamino-methylidene)amino)-3H-1,2,4-dithiazole-3-thione or DDTT, obtained from GlenResearch, 22825 Davis Drive, Sterling, VA 20164) was dissolved in 3:2 (v/v) pyridine/acetonitrile (0.05 M) and thiolation time was 5 min in all cycles. Cap A (acetic anhydride/tetrahydrofuran 9.1:90.9 v/v) and Cap B (tetrahydrofuran/N-methylimidazole/pyridine 8:1:1 v/v/v) were mixed in situ in a 1:1 v/v ratio for capping. Cyanoethyl backbone removal was performed with diethylamine/acetonitrile (10% v/v) after a final 5′-detritylation. Oligonucleotides were cleaved from the solid support by four treatments (5-10-10-10 min) with AMA (ammonium hydroxide 26%:methylamine 40% 1:1) at 20° C. and further deprotected by treatment at 65° C. for 1 h in the same solution. Oligonucleotides were dried under reduced pressure at 45° C. for 2 h and subsequently diluted in sodium acetate buffer (0.3 M, 330 piL, pH 5.5). Ice-cold ethanol (4-5 volumes) was then added and the oligonucleotides were precipitated at −20° C. for 2 h. Suspensions were centrifuged (18000 rcf) for 10 min before the supernatant was discarded. Oligonucleotide pellets were dried under reduced pressure at 45° C. for 30 min and used as such without further purification.

The ASO described herein are all 3-10-3 gapmers with LNA-DNA-LNA structure, with the LNA termini being constructed from LNA-A, LNA-5Me-C, LNA-G & LNA-T building blocks and the central DNA unit being constructed from deoxy-A, deoxy-5Me-C, deoxy-G & deoxy-T (see definitions directly below).

Amidites used in ASO synthesis:
deoxy-A: (2R,3S,5R)-5-(6-benzamido-9H-purin-9-yl)-2-((bis(4-methoxyphenyl)(phenyl)methoxy) methyl)tetrahydrofuran-3-yl (2-cyanoethyl) diisopropylphosphoramidite;
deoxy-5Me-C: (2R,3S,5R)-5-(4-benzamido-5-methyl-2-oxopyrimidin-1(2H)-yl)-2-((bis(4-methoxyphenyl)(phenyl)methoxy)methyl)tetrahydrofuran-3-yl (2-cyanoethyl) diisopropylphosphoramidite;
deoxy-G: (2R,3S,5R)-2-((bis(4-methoxyphenyl)(phenyl)methoxy)methyl)-5-(2-isobutyramido-6-oxo-1,6-dihydro-9H-purin-9-yl)tetrahydrofuran-3-yl (2-cyanoethyl) diisopropylphosphoramidite;
deoxy-T: (2R,3S,5R)-2-((bis(4-methoxyphenyl)(phenyl)methoxy)methyl)-5-(5-methyl-2,4-dioxo-3,4-dihydropyrimidin-1(2H)-yl)tetrahydrofuran-3-yl (2-cyanoethyl) diisopropylphosphoramidite;
LNA-A: (1R,3R,4R,7S)-3-(6-benzamido-9H-purin-9-yl)-1-((bis(4-methoxyphenyl)(phenyl)methoxy) methyl)-2,5-dioxabicyclo[2.2.1]heptan-7-yl (2-cyanoethyl) diisopropylphosphoramidite;
LNA-5Me-C: (1R,3R,4R,7S)-3-(4-benzamido-5-methyl-2-oxopyrimidin-1(2H)-yl)-1-((bis(4-methoxyphenyl)(phenyl)methoxy)methyl)-2,5-dioxabicyclo[2.2.1]heptan-7-yl (2-cyanoethyl) diisopropylphosphoramidite;
LNA-G: (1R,3R,4R,7S)-1-((bis(4-methoxyphenyl)(phenyl)methoxy)methyl)-3-(2-isobutyramido-6-oxo-1,6-dihydro-9H-purin-9-yl)-2,5-dioxabicyclo[2.2.1]heptan-7-yl (2-cyanoethyl) diisopropylphosphoramidite; and
LNA-T: (1R,3R,4R,7S)-1-((bis(4-methoxyphenyl)(phenyl)methoxy)methyl)-3-(5-methyl-2,4-dioxo-3,4-dihydropyrimidin-1(2H)-yl)-2,5-dioxabicyclo[2.2.1]heptan-7-yl (2-cyanoethyl) diisopropylphosphoramidite.

### Human ARHGEF6 ASO Spot Test

THP-1 cells endogenously expressing human ARHGEF6 were used to determine knock down efficacy of ARHGEF6 ASOs. ASOs were tested in biological duplicates at a single concentration, and analyses of relative ARHGEF6 expression levels were performed in technical triplicates.

ASOs were pre-dispensed into 96-well cell culture plates with an Echo 655 Acoustic Liquid Handler (Labcyte) for a final concentration of 3.5 μM. Cryo-preserved THP-1 cells (ATCC® TIB-202™) were defrosted according to standard procedures, washed in culture medium (RPMI 1640 with GlutaMax, 2 g/L glucose, HEPES, MEM Non-Essential Amino Acids, 1 mM sodium pyruvate, 10% FBS and 50 μM β-mercaptoethanol) and plated on top of ASOs at a density of 3.5×10⁴ viable cells per well in 100 μL culture medium. Cells were incubated at 37° C., 5% CO₂ for 24 hours.

After incubation, cells were transferred to 96-well V-bottom polypropylene microplates and pelleted at 500 g for 3 minutes. Medium was removed and cells lysed in 20 μL lysis buffer (Qiagen RNeasy RLN lysis buffer with 4% RNAsecure™ RNase Inactivation Reagent) for 5 minutes at room temperature. 2 μL of lysates were used as templates in 20 μL-reverse transcription (RT) reactions (50% RT buffer and 5% enzyme mix from Invitrogen's Cells-to-CT Bulk RT Reagents), and RT was performed at 37° C. for 60 min, then 95° C. for 5 min.

The cDNA samples were diluted 1:4 and Real-Time PCR reactions were set up using 3 μL cDNA, TaqMan™ Fast Advanced Master Mix, and ARHGEF6 or Hypoxanthine Phosphoribosyltransferase 1 (HPRT1) TaqMan™ Gene Expression Assays (Hs00374477_m1 and Hs02800695_m1, all Applied Biosystems) in a total volume of 10 μL. Amplifications were performed on a QuantStudio™ 7 Flex Real-Time PCR System (Applied Biosystems) and were conducted at 50° C. for 2 min, 95° C. for 10 min, followed by 40 cycles of 95° C. for 15 s and 60° C. for 1 min. Quantification cycle (Cq) values were determined by the software using the Auto Baseline and Auto Threshold options and were then used to calculate relative ARHGEF6 expression (2\hat{ }-dCq) normalized against the reference gene HPRT1.

ARHGEF6 ASO Concentration-Response Curves in Podocytes ARHGEF6 ASO potency was assessed in human iPS-cell derived podocytes endogenously expressing human ARHGEF6. The iPS cells were derived from the human fibroblast cell line BJ (ATCC CRL-2522) using an mRNA reprogramming kit from Stemgent, followed by targeted integration of a Tet-On-regulated Cas9 transgene (see Lundin, A., Porritt, M. J., Jaiswal, H. et al. Development of an ObLiGaRe Doxycycline Inducible Cas9 system for pre-clinical cancer drug discovery. Nat. Commun. 11, 4903 (2020) for details). Differentiation was then performed based on a published protocol (see Musah, S., Dimitrakakis, N., Camacho, D. M. et al. Directed differentiation of human induced pluripotent stem cells into mature kidney podocytes and establishment of a Glomerulus Chip. Nat. Protoc. 13, 1662-1685 (2018)). ASOs were tested in serial dilutions in biological duplicates, and analyses of relative ARHGEF6 expression levels were performed in technical triplicates.

Human iPS-cell derived podocytes seeded at a density of 3.5×10⁴ viable cells per cm² before podocyte maturation, were maintained in complete serum-free RocketFuel™ maintenance medium (Cell Systems, Kirkland, WA 98034, USA, SF-4Z0-50). Cells were treated with serial 3-fold dilutions of ASOs for 24 hours at 37° C., 5% CO₂, and were then lysed in 20 μL lysis buffer and processed according to the same protocol as described for THP-1 cells above. Relative ARHGEF6 expression (2\hat{ }-dCq) was normalized against the reference gene HPRT1, and data were analyzed and concentration-response curves plotted using GraphPad Prism version 8.0.1 for Windows (GraphPad Software Inc., La Jolla, CA, USA; www.graphpad.com). IC₅₀s were calculated with a four-parameter logistic fit using the equation

$$Y = Bottom + ( Top - Bottom ) / ( 1 + 10 \hat{} ( ( Log {IC}_{50} - X ) * HillSlope ) )$$

where Y is the response, X is the base 10 logarithm of the ASO concentration, Bottom corresponds to the maximum reduction achieved, and Top is the lowest level achieved.

### Protein Production (Expression and Protein Purification)

Expression of the N-terminally HN-tagged ARHGEF6 SH3-DH-PH domain construct (N-6×HN-GGG-TEV-hARHGEF6 (M155-A551)) and the PAK1 construct (6HN-GGG-TEV-PAK1(M1-K269)) were carried out in bacteria (E. coli strain BL21XDE3Gold) using autoinduction methodologies.

The ARHGEF6 full length construct (N-6×HN-GGG-TEV-hARHGEF6(M1-P776) and construct ARHGEF6(M155-P776)-TEV-GSG-HALO-6His were expressed in insect cells (Sf21 cells) with a time of harvest (TOI) of 48h.

For all constructs cells were harvested by centrifugation and stored at −80° C. The same purification method was used for all constructs, with processes differing only in the cell lysis method.

The cells were resuspended in buffer (50 mM Tris/HCl, pH 8, 300 mM NaCl, 10% glycerol, 1 mM TCEP, and 1× Complete protease inhibitors) prior to lysis. The ARHGEF6 SH3-DH-PH domain construct and the PAK1 construct (6HN-GGG-TEV-PAK1(M1-K269)) were lysed by high pressure homogenisation (Emulsiflex), the ARHGEF6 full length construct and the ARHGEF6(M155-P776)-TEV-GSG-HALO-6His construct were lysed by the use of an Ultra-Turrax. After clarification by centrifugation and addition of imidazole to a final concentration of 20 mM, the lysates were loaded on to a 5 ml Ni HisTrap column (Cytiva) equilibrated in a buffer consisting of 50 mM Tris/HCl, pH 8, 500 mM NaCl, 10% glycerol, 1 mM TCEP, 20 mM imidazole and eluted with buffer consisting of 50 mM Tris/HCl, pH 8, 500 mM NaCl, 10% glycerol, 1 mM TCEP, 500 mM imidazole. Fractions containing the target protein peak fractions were pooled.

A size exclusion column (Superdex200, Cytvia) was used as second purification step in buffer 50 mM Tris/HCl, pH 8, 150 mM NaCl, 10% glycerol, 1 mM TCEP. The protein peak was collected, concentrated and stored in −80° C.

The GIT1 construct, GIT1-TEV-6×His, was expressed in bacteria (E. coli), harvested by centrifugation and stored at −80° C.

The cells were lysed with sonication in buffer 20 mM Tris-HCl, pH 7.4, 250 mM NaCl, 5 mM imidazole, protease inhibitors. After clarification by centrifugation, the lysate was mixed with cobalt resin equilibrated in a buffer consisting of 20 mM Tris-HCl, pH 7.4, 250 mM NaCl, 5 mM imidazole. After incubation in batch mode, the resin was placed into an empty column and eluted with buffer consisting of 20 mM Tris-HCl, pH 7.4, 250 mM NaCl, 500 mM imidazole. Fractions containing the target protein peak fractions were pooled.

A size exclusion column (Superdex200, Cytvia) was used as second purification step in buffer 50 mM Tris-HCl, pH 8, 300 mM NaCl, 1 mM TCEP, 1% Glycerol. The protein peak was collected, concentrated and stored at −80° C.

Preparation of Biotinylated hARHGEF6-Isoform2

Biotinylation of full-length hARHGEF6-isoform2 with C-terminal HALO tag (hARHGEF6(M155-P776)-TEV-GSG-HALO-AAA-6×His) was carried out by mixing 42 μM protein with 50 μM HaloTag® PEG-Biotin Ligand (Promega G859A) in buffer containing 50 mM Tris/HCl, pH 8, 150 mM NaCl, 10% glycerol, 1 mM TCEP. Reaction was incubated at room temperature for 2 hrs and the protein was then purified gel-filtration over Superose-6 increase 3.2/300 column in buffer containing 50 mM Tris/HCl, pH 7.5, 250 mM NaCl, 10% glycerol, 1 mM DTT).

### Surface Plasmon Resonance (SPR) Binding

SPR binding experiments on the HN-tagged ARHGEF6 SH3-DH-PH domain construct (N-6×HN-GGG-TEV-hARHGEF6 (M155-A551)) as well as the HN-tagged full-length ARHGEF6 construct were performed on a Biacore T200 optical biosensor unit (GE Healthcare) at 20° C. Sensor chips Series S NTA (Research grade, GE Healthcare) were equilibrated at room temperature prior to use. The running buffer for protein tethering and subsequent ligand binding experiments was 10 mM HEPES, 150 mM NaCl, 1 mM TCEP, 0.05% P20, pH 7.40. The tethering of the protein was performed at a flow-rate of 10 μl min⁻¹. The NTA surface was conditioned by a one-minute injection of a 350 mM EDTA solution (pH 8.3) followed by a one-minute injection of running buffer supplemented with 0.5 mM NiCl₂. To allow for a covalent tethering after the initial capture step, the conditioned surface was activated for 7 min with 50 mM NHS and 200 mM EDC. This was immediately followed by an injection of protein in running buffer at a concentration of 50-100 μg ml⁻¹ with a contact time of 2-3 min resulting in final coupling densities of 5500-6000 RU for the ARHGEF6 SH3-DH-PH domain construct and 7000-7500 RU for the full-length ARHGEF6 construct. The deactivation of residual esters was achieved through a 7 min injection of 300 mM Ethanolamine in running buffer. Reference surfaces were prepared accordingly, omitting the injection of protein over the activated reference surface.

The SPR binding experiments on the biotinylated ARHGEF6-isoform2 were performed on a Biacore S200 optical biosensor unit (GE Healthcare) at 20° C. Sensor chips Series S HLC30M (Research grade, GE Healthcare) were equilibrated at room temperature prior to use. The running buffer for protein tethering and subsequent ligand binding experiments was 20 mM HEPES, 250 mM NaCl, 1 mM TCEP, 0.05% P20, pH 7.5. The surface was conditioned with 50 mM NaOH and 1 M NaCl and activated for 10 min with 50 mM NHS and 200 mM EDC at a flow rate of 10 μl min⁻¹. This was immediately followed by an injection of 10 μg/ml neutravidin in 10 mM sodium acetate pH 5.5 for 240 s resulting in coupling density of nearly 1000 RU. The deactivation of residual esters was achieved through a 5 min injection of 1 M Ethanolamine pH 8.5. Biotinylated ARHGEF6-isoform2 (65 nM) was then injected at a lower flow-rate of 2 μl min⁻¹ for several minutes resulting in the desired coupling density and the remaining neutravidin binding sites were quenched by injecting 1 mM biotin in running buffer for 1 min at a flow rate of 10 μl min⁻¹. A range of ligand coupling densities 300-1200 RU were tested.

The binding experiments with PAK1 peptide were performed at a flow rate of 30 μl min⁻¹ and by employing the method of multi-cycle kinetics. A contact time of 45 s was selected, which was followed by a 2 min dissociation phase. The PAK1 peptide (MW: 2562.8 Da) was dissolved in DMSO to 50 mM and a digital dispenser HP D300 (Tecan) was used to set up a compound concentration series using 8 concentrations with a 2-fold dilution pattern. The tested concentrations were 400, 800, 1600, 3200, 6400, 12800, 25600 and 51200 nM. Prior to the analysis of compound binding, three running buffer blanks were injected to equilibrate the instrument. The data collection rate was set to 10 Hz and all experiments were repeated three times to allow for error estimations. Due to the low DMSO mismatch (maximum around 0.1%) introduced by the compound addition, no solvent correction was required.

The binding experiments with PAK1 were also performed at a flow rate of 30 μl min⁻¹ by employing the method of multi-cycle kinetics. A range of concentration of PAK1 (1.95 to 125 nM) were prepared in running buffer and were injected with a contact time of 1 min and dissociation time of 10 mins for each concentration.

Experiments with GIT1 were performed using the method of single-cycle kinetics at the flow-rate of 30 μl min⁻¹. A range of concentrations of GIT1 (2 to 500 nM) were prepared in running buffer and were consecutively injected with a contact time of 10 min for each concentration followed by dissociation time of 30-60 mins at the end.

For all experiments, the reference-subtracted data was further analysed by subtracting a similar experiment containing only buffer injections in order to correct for injection artefacts, systematic noise and instrument drift. This double-referenced data was fitted either using a 1:1 kinetic interaction model or a steady-state fit accounting for the transient binding process in order to extract both kinetic and affinity data.

Isothermal titration calorimetry (ITC) binding. The SPR binding experiments on the HN-tagged ARHGEF6 SH3-DH-PH domain construct (N-6×HN-GGG-TEV-hARHGEF6 (M155-A551)) were performed on a MicroCal Auto-iTC200 unit (Malvern) at 25° C. The titration buffer used for the ligand binding experiments was composed of 10 mM HEPES, 150 mM NaCl, 1 mM TCEP, 0.05% P20, 1% DMSO, pH 7.40.

The protein was passed over a PD10 column (GE Healthcare) pre-equilibrated with titration buffer according to manufacturer's instructions and adjusted to a final concentration of 25-30 mM. The PAK1 peptide (MW: 2562.8 Da) was dissolved in DMSO to 30 mM and diluted 1:100 into titration buffer without DMSO in order to achieve a nominal concentration of 300 mM in a buffer matching exactly the composition of the titration buffer. The titration experiment was started by injecting 1×0.4 ml of the PAK1 peptide solution into the protein solution followed by injecting 19×2 ml after a 60s waiting period. The spacing between the individual injections was set to 90s, while a 5s filtering period and a high feedback mode was applied to enable rapid equilibration after each injection.

### Data Fitting of ITC Binding Experiments

Prior to the fitting of the data, the data was treated by subtracting a similar experiment containing only buffer injections in order to correct for injection artefacts, systematic noise and instrument drift.

The data has been fitted using a 1:1 interaction model and revealed a K_{D} value of 4.26 μM with a stoichiometry value of 0.87. Under the experimental conditions, the binding appears to be strongly enthalpically driven with a DH value of −21.9 kcal/mol.

### BTBR Ob/Ob Mouse Model

### Model Description

The leptin-deficient BTBR ob/ob mouse (homozygous for the spontaneous mutation, Lep^{ob}) exhibit extreme obesity due to hyperphagia. They develop severe and progressive hyperglycemia and hypertriglyceridemia, elevated plasma insulin, impaired wound healing and are also hypometabolic and hypothermic (see e.g. K. L Hudkins et al, J Am Soc Nephrol 2010, Sep, 21(9), 1533-42).

### Translation to Human Kidney Injury Mechanisms

The diabetic BTBR ob/ob mouse model mimics key features of early diabetic nephropathy (DN) in humans (CKD/DN stage 2 in man), with chronic injury limited to glomeruli. They also develop severe progressive albumin and proteinuria and some of the morphological features typical for human DN, such as mesangial expansion, basement membrane thickening, and, to some extent, mesangiolysis. The BTBR ob/ob mice are hyperfiltrating, thereby resembling early stages of human DN.

### Study Design

The study design for each mouse is outlined in FIG. 13. BTBR ob/ob and BTBR wild-type (wt) females were used in the study (embryos from BTBRV(B6)-Lep^{ob}/WiscJ stock no. 004824 provided by The Jackson Laboratory and bred at AstraZeneca R&D breeding facility, Gothenburg, Sweden). From 0-6 weeks of age, a standard chow diet was used (R36, Lantmännen, Stockholm, Sweden. Energy percentage of 18.5% protein, 4% fat, and 55.7% nitrogen-free extract and with a total energy content of 3011 kcal/kg).

### Husbandry, Housing, Light Cycle, Food

The mice were maintained in a controlled room environment with temperature 21±0.5° C., 12:12-h light-dark cycle (lights on at 06:00 am) and relative humidity 50±5%. The mice were housed in transparent Makrolon cages with wooden bedding and nesting material. The cages were placed on partial heating pads to maintain body temperature. The mice were caged in groups of 2-3 individuals with free access to water and standard chow diet (R3, Lantmännen, Stockholm, Sweden. Energy percentage of 21% protein, 5% fat and 51.5% Nitrogen-free extract and with a total energy content of 3011 kcal/kg). The mice were on the R3 diet from 6- to 20 weeks of age.

### Ethical Approval

Experimental procedures were approved (Idnr: 002668, Dnr: 5.8.18-04150/2020) by the Regional Laboratory Animal Ethics Committee of Gothenburg, Sweden. The holding facility has received full accreditation from the Association for Assessment and Accreditation of Laboratory Animal Care (AAALAC).

### Randomisation

The mice were randomised at 7 weeks of age on data based on urinary albumin-to-creatinine ratio (UACR), body weight and 3 hrs fasting glucose. Glucose was measured on tail vein blood sample with a standard glucometer (Accu-Chek mobile®)

### Dosing Regimen and Route

All BTBR ob/ob mice were dosed a volume of 3 ml/kg intraperitoneally (ip) once weekly according to Table 3. A group of non-injected healthy BTBR wt mice was included as healthy control for comparative purposes.

TABLE 3
Group allocation in BTBR ob/ob ASO study

| | Treatment | Group | N |
|---|---|---|---|
| 1. | Disease Placebo control (PBS injected) | BTBR ob/ob | 12 |
| 2. | ASO 9, 1 mg/kg | BTBR ob/ob | 10 |
| 3. | ASO 9, 8 mg/kg | BTBR ob/ob | 11 |
| 4. | Healthy control, not injected | BTBR wt | 13 |

### Experimental Procedures

Urine collection: The mice were placed in an empty cage for a maximum of 1 hour with water ad libitum and spontaneous urination was collected. Urine samples were collected throughout the study for analyses at 7, 10, 12, 14, 16, 18, and 19 weeks of age.

Body weight measurement: Body weight was recorded once weekly prior to dosing.

Food and water intake measurement: A 24-hour food and water intake measurement was recorded per cage once a week throughout the whole study.

Termination and organ sampling: Mice were fasted 3-5 hrs before termination (20 weeks of age). All mice were anesthetized with isoflurane [Datex Ohmeda Isotec 5 Isoflurane Anesthesia Vaporizer (5% isoflurane, 2 L/min air)], and after loss of conscious and muscle tonus, they were decapitated for terminal blood collection.

Plasma collection: Blood samples were collected in EDTA coated tubes, and centrifuged at 12600×g for 2 min Plasma was collected and stored at −80° C. until analysis.

Tissue weight: At endpoint, kidneys, quadriceps muscle, subcutaneous adipose tissue, heart, spleen and liver were collected for subsequent analyses.

### UACR Measurement

### Albumin:

Urinary albumin was measured using a competitive antibody capture ELISA, where the anti-albumin antibody is conjugated to horseradish peroxidase, utilizing a commercially available kit (Catalogue number 1011, Ethos Bioscience, NJ, USA), following manufacturer's instruction. Briefly, sample and anti-mouse albumin AB-HRP were added to mouse albumin-coated well. Plates were washed to remove unbound Ab-HRP-Albumin from the fluid phase of the well. The bound antibody-conjugate (bound to the albumin of the stationary phase) was detected using tetramethylbenzidine (TMB) in a chromogenic reaction. The reaction was stopped with acid, and absorbance was measured at 450 nm. The background absorbance was measured at 570 nm.

### Creatinine:

Urinary creatinine was measured using a commercially available kit (ab65340; Abcam®, Cambridge, MA, USA), where creatinine is converted to creatine by creatininase. Then, creatine was converted to sarcosine, which was specifically oxidized to produce a product which reacted with a probe to generate a red color. In this assay we took both colorimetric and fluorometric measurement to encompass a wider range of concentration. Samples and standards were added with a background mix containing creatinase, probe, and an ‘enzyme mix’ and incubated at 37° C. for an hour in a half-area plate (Corning, catalogue no 3695), followed by a background readout for both absorbance (OD=570 nm) and fluorescence (Ex/Em=535/595 nm). Then, a reaction mix containing creatininase was added and the plate was incubated for an hour at 37° C. A final measurement was taken at both optical density (OD=570 nm) and fluorescence (Ex/Em=535/595 nm).

RNA Extraction, cDNA Synthesis, and Quantitative RT-PCR
RNA Extraction from Mouse Kidney, and cDNA Synthesis

Total RNA was extracted from cells using RNeasy Mini kit (QIAGEN), while for mouse glomeruli, total RNA was extracted using RNeasy 96 kit following manufacturer's instruction (Qiagen). cDNA was synthesized using High-capacity cDNA reverse transcription kit following manufacturer's instruction (Applied Biosystem).

Real-Time qPCR

RT-qPCR was performed using TaqMan™ Fast Advanced Master Mix on QuantStudio™ 7 Flex Real-Time PCR System (Applied Biosystems). Mouse ARHGEF6, Nphs1, Nphs2, Wt1, Synpo, MafB and Hprt TaqMan™ Gene Expression Assays (Mm00461751_m1, Mm01176615_g1, Mm01292252_m1, Mm01337048_m1, Mm03413333_m1, Mm00627481_s1 and Mm03024075_m1) were purchased from Applied Biosystem.

### Kidney Histology

Kidney obtained at termination of the studies described above were transversely cut and fixated for 48 h in 4% formaldehyde solution, dehydrated, paraffin embedded, and cut at 2 μm thin sections. Sections were deparaffinized, rehydrated and stained with routine Periodic Acid Shiff (PAS) in a Leica ST5020-CV5030 automated stainer. Whole kidney sections were scanned on a Pannoramic scan II (3DHlstec Ltd, Hungary) scanner at 20× magnification.

### Protein Extraction and LC/MS

Mouse kidney tissues were homogenized in lysis buffer (RIPA buffer, nuclease, protease inhibitor) using metal beads and a tissue lyzer (Qiagen). Protein lysates were subjected to reduction and alkylation, and subsequent protease treatment using Trypsin/LysC. Peptide samples were spiked with a corresponding heavy labelled AQUA peptide to enable absolute quantification. LC-MS/MS analysis was conducted on a Fusion Lumos (Thermo Fisher Scientific) coupled with an Evosep LC-system and nano-electrospray ion source (Easy Spray Source, Thermo Fisher Scientific). The MS was operated in parallel-reaction-monitoring (PRM) mode, targeting the endogenous and heavy labelled peptide of ARHGEF6. Data analysis was performed using Skyline.

### 3D Structured Illumination Microscopy (3D-SIM) Analysis by NIPOKA

Formalin-fixed, paraffin embedded (FFPE) kidney sections from 3 BTBR wt, 6 BTBR ob/ob and 6 BTBR ob/ob+ASO 9 (8 mg/kg) were sent to NIPOKA GmbH (Greifswald, Germany) for analysis. The sections were double stained for integrin α3 (podocyte foot processes) and nephrin (slit diaphragms) and visualized by Structured Illumination Microscopy (SIM) using an N-SIM super-resolution microscope (Nikon, Tokyo, Japan) with a 100× silicone objective (ref: PMID 35884965). A 3D SIM reconstruction was made using NIS-Elements AR 5.30 software for 20 glomeruli per mouse. Filtration slit density (FSD) for a selected capillary area (A) was determined by dividing the filtration slit length within the selected area (ISD) by the size of the area (ISD/A). The same analysis without quantification was done on samples stained for Ehd3 and synaptopodin, with a qualitative assessment of the glomerular endothelial ultrastructure done by NIPOKA in a blinded fashion.

### Exposure

### Sample Homogenization:

Mouse tissue samples and blank samples were weighed and transferred into 2 mL Precellys tubes (Bertin Corp) containing zirconium oxide beads (2.8 mm, NETZSCH), kept cool with ice. Cold Milli-Q water (Merck, Q-POD) was added to each tube, followed by homogenization using a homogenizer (Precellys Evolution, Bertin) at 5500 rpm for two cycles of 25 seconds each, with 2-minute intervals on ice between cycles. This process was repeated until samples reach the desired homogeneity.

### Sample Extraction:

A liquid-liquid extraction protocol was executed utilizing the Agilent Bravo liquid handling automation system for the extraction of anti-sense oligonucleotides (ASOs) from samples. Standard curves were generated by spiking a stock solution of ASOs into blank tissue homogenate followed by serial dilution. 100 μL of standards and study samples were pipetted into a 1 mL Nunc™ 96 DeepWell polypropylene plate, Natural RNase/DNase-Free (REF 260252, Thermo Scientific). Milli-Q water and ammonium hydroxide (28%-30%, Sigma-Aldrich) were added to adjust the pH. The phenol-chloroform-isoamyl alcohol mixture (Sigma-Aldrich) was added to the samples, followed by pipette-mixing for 50 cycles until phase separation was achieved. Subsequently, the plate was centrifuged for 10 minutes at 4000 rpm using the Eppendorf Centrifuge 5810R at 4° C. The aqueous phase in each well was then transferred to a new Nunc plate, and 1,2-dichloroethane (Thermo Scientific) was added. The mixture was pipette-mixed for 25 cycles and then centrifuged again for 10 minutes at 4000 rpm at 4° C. The aqueous phase was transferred to a new Nunc plate and loaded into an evaporator (MiniVap, Porvair Sciences) under a gentle flow of nitrogen gas overnight to ensure thorough drying. Upon complete evaporation, 100 μL of Milli-Q water was added to reconstitute the samples, followed by vortexing at 1500 rpm for 5 minutes to ensure homogeneity. Subsequently, the samples were further diluted to a range of 10 to 40 times with a 5 nM internal standard solution in a new Nunc plate. The resulting plate was then prepared for LC-MS/MS injection.

### LC-MS/MS Quantification:

For accurate quantification of anti-sense oligonucleotides, Waters Xevo TQ-XS triple quadrupole mass spectrometry instrument was employed. Ion-pair reverse-phase mobile phases were prepared, consisting of mobile phase A comprising 200 mM 1,1,1,3,3,3-Hexafluoroisopropanol (TCI) and 7.5 mM triethylamine (Sigma-Aldrich) in Milli-Q water, while mobile phase B consisted of methanol (Merck Supelco hypergrade for LC-MS). A 13-minute HPLC method was devised for eluting the anti-sense oligonucleotide, wherein mobile phase B increased from 15% to 30% over 6 minutes. The flow rate was set at 0.3 mL/min, and the column temperature was maintained at 60° C. using an Acquity Premier Oligonucleotide BEH C18 column. The multiple reaction monitoring (MRM) transitions monitored for AZ14294632 were 757.04→94.83 and for AZ14283945 were 764.47→94.83, with the internal standard being 764.48→97.1. During the chromatographic run, the LC flow was directed to waste for the initial 4.5 minutes, switched to the mass spectrometry from 4.5 to 9 minutes, and then redirected to waste to prevent instrument contamination.

### Results and Data Interpretation

### Downregulation of ARHGEF6

FIG. 14 shows the ASO 9 knock-down effect on ARHGEF6 gene and protein expression in mouse kidney. A dose dependent downregulation of ARHGEF6 is observed with a 58% knock-down being achieved with 8 mg/kg ASO 9 treatment.

### Improved UACR

As can be seen in FIG. 15, UACR decreases significantly in mice treated with 8 mg/kg of ASO 9. A significant improvement in renal damage after 8 weeks on treatment relative to control was observed, and this 61% improvement window is maintained until the end of the experiment. The data for the 1 mg/kg dose of ASO 9 also suggest a positive trend, albeit no statistically significant difference in results between this dose cohort and the disease control was obtained in the present experiment.

### Histological Improvements

To further confirm that ASO 9 treatment improves glomerular health, kidney glomerular score was quantified by a trained AI algorithm. This tool assigns a numerical score to each glomerulus in an entire histological kidney section, providing an indication of glomerular damage. The definitions are 0: Normal; No or minimal changes, 1: Mild; slight-moderate mesangial matrix expansion with <4 mesangial cells/glomerular segment, 2: Moderate; moderate mesangial matrix expansion with 4-6 mesangial cells/glomerular segment, 3: Severe; moderate to severe mesangial matrix expansion with >6 mesangial cells/glomerular segment.

Treatment with 8 mg/kg of ASO 9, significantly improved glomerular score, in the fractions shown in FIG. 16, we can observe that the amount of severely injured glomeruli is reduced while the number of normal glomeruli is increased (there were almost non-existent normal glomeruli in the BTBR ob/ob control mice treated with placebo).

Improvement in glomerular health was confirmed using super resolution microscopy. Restoration of the podocyte foot process was shown by elevation of the filtration slit density (FSD) to the levels observed in healthy mice in 4 out of 6 animals receiving ASO 9 treatment (see FIG. 17A and FIG. 17B). ASO 9 treatment also improved glomerular diameter (see FIG. 17B) aligning with the improvement of glomerular score seen in FIG. 16. Qualitative data also showed glomerular endothelial improvements by reduction of endothelial protrusions with ASO 9 in BTBR ob/ob resembling the healthy endothelial phenotype seen in BTBR wt mice (FIG. 18, arrow indicates green stained endothelium with structure in ASO 9 treated mice resembling closely those in BTBR wt mice).

### Plasma AST and ALT

The levels of Aspartate aminotransferase (AST) and alanine aminotransferase (ALT), the two liver enzymes used in healthcare as a biomarker for liver damage were not increased with ASO 9 treatment compared to BTBR ob/ob disease control mice (FIG. 19).

### Body Weight and Organ Weights

Treatment with ASO 9 did not have any effect in body weight or organ weight (see FIG. 20).

### Tissue Exposure

FIG. 21 shows the level of ASO 9 in different organs at the end of the 12-week treatment. The amounts of ASO 9 found in kidney are ~35 times bigger that in the other organs (liver, skeletal muscle and heart) indicating that ASO level found at the intended site of action far surpass those in other tissues.

1. An ARHGEF6 specific antisense oligonucleotide for use in the treatment or prophylaxis of chronic kidney disease.

2. An ARHGEF6 specific antisense oligonucleotide for use according to claim 1 that is a human ARHGEF6 specific antisense oligonucleotide.

3. An ARHGEF6 specific antisense oligonucleotide for use according to claim 1 or claim 2, having a gapmer structure 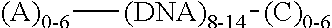
in which:
(A)₀₋₆ and (C)₀₋₆ independently denote units comprising from 0 to 6 modified ribonucleic acid monomers;
(DNA)₈₋₁₄ denotes a unit comprising from 8 to 14 2-deoxyribonucleic acid monomers;
wherein the subscript denotes the number of monomers from which A, B and C are constituted.

4. An ARHGEF6 specific antisense oligonucleotide for use according to claim 3 that is an ARHGEF6 ASO with a 3-10-3 LNA-DNA-LNA gapmer structure.

5. An ARHGEF6 specific antisense oligonucleotide for use according to any preceding claim, produced by a process comprising the steps of a) selecting a ASO that targets an accessible region of ARHGEF6, optionally a region of ARHGEF6 having an accessibility score>0.001 as determined using the Vienna RNA RNAplfold algorithm; b) determining that the candidate ASO has perfect complementarity to only the ARHGEF6 sequence; c) determining that the candidate ASO has complementarity with 1 mismatch to no greater than 50 other genes; d) filtering to remove ASOs that target a region with a minor allele frequency of >0.05, and e) further filtering to remove ASOs that have CG motifs and has a % GC content of <10

6. An ARHGEF6 specific antisense oligonucleotide for use according to any of claims 1 to 5, wherein the use is for a patient diagnosed as having DN (diabetic nephropathy), FSGS (focal segmental glomerulosclerosis), HTN (renal hypertension), IgAN (IgA nephropathy/Berger's disease), RPGN (rapidly progressive glomerulonephritis) and SLE (systemic lupus erythematosus) but not MCD (minimal change disease), MGN (membranous glomerulonephritis) and TMD (thin glomerular basement membrane disease).

7. A method of treatment or prophylaxis comprising administering an effective amount of an ARHGEF6 specific antisense oligonucleotide to a patient in need thereof, wherein the patient in need thereof has chronic kidney disease or has been identified as being at risk of developing chronic kidney disease.

8. A method of treatment or prophylaxis according to claim 7 wherein the patient in need has been diagnosed as having DN (diabetic nephropathy), FSGS (focal segmental glomerulosclerosis), HTN (renal hypertension), IgAN (IgA nephropathy/Berger's disease), RPGN (rapidly progressive glomerulonephritis) and SLE (systemic lupus erythematosus) but not MCD (minimal change disease), MGN (membranous glomerulonephritis) and TMD (thin glomerular basement membrane disease).

9. An ARHGEF6 specific antisense oligonucleotide for use in the manufacture of a medicament for the treatment or prophylaxis of chronic kidney disease.

10. An ARHGEF6 specific antisense oligonucleotide for use in the manufacture of a medicament according to claim 9, wherein the intended use is for a patient diagnosed as having DN (diabetic nephropathy), FSGS (focal segmental glomerulosclerosis), HTN (renal hypertension), IgAN (IgA nephropathy/Berger's disease), RPGN (rapidly progressive glomerulonephritis) and SLE (systemic lupus erythematosus) but not MCD (minimal change disease), MGN (membranous glomerulonephritis) and TMD (thin glomerular basement membrane disease).

11. An ARHGEF6 specific antisense oligonucleotide for use according to claims 1 to 6, for use in the manufacture of a medicament according to claims 9 or 10, or method of treatment according to claims 7 or 8, wherein the use or the method of treatment is indicated on the basis of measured GFR, eGFR or UACR obtained from a sample from a patient that indicates the patient has chronic kidney disease.

12. An ARHGEF6 specific antisense oligonucleotide for use according to claims 1 to 6, for use in the manufacture of a medicament according to claims 9 or 10, or method of treatment according to claims 7 or 8, wherein the use is for a patient with a measured eGFR or GFR of <70 mL/min/1.73 m² or with a measured UACR is >30 mg/g.

13. A process for producing an ARHGEF6 specific antisense oligonucleotide comprising the steps of:
a) selecting an antisense oligonucleotide that targets an accessible region of ARHGEF6, optionally a region of ARHGEF6 having an accessibility score>0.001 as determined using the Vienna RNA RNAplfold algorithm;
b) determining that the candidate antisense oligonucleotide has perfect complementarity to only the ARHGEF6 sequence;
c) determining that the candidate antisense oligonucleotide has complementarity with 1 mismatch to no greater than 50 other genes;
d) filtering to ensure that the candidate antisense oligonucleotide does not target a region with a minor allele frequency of >0.05;
e) ensuring that the candidate antisense oligonucleotide has no CG motifs and has a % GC content of >10; and
f) synthesising the resultant oligonucleotide.

14. A human ARGHEF6 specific antisense oligonucleotide produced by a process according to claim 13.


SEQUENCE LISTING (17 sequences)

SEQ ID NO: 1 - DNA, 16 bp, synthetic construct
  source 1..16: mol_type = other DNA
gtattagcagcactaa

SEQ ID NO: 2 - DNA, 16 bp, synthetic construct
  source 1..16: mol_type = other DNA
tgtaccatgtagctat

SEQ ID NO: 3 - DNA, 16 bp, synthetic construct
  source 1..16: mol_type = other DNA
aatgtgcatacttcct

SEQ ID NO: 4 - DNA, 16 bp, synthetic construct
  source 1..16: mol_type = other DNA
gatggtttgcacagta

SEQ ID NO: 5 - DNA, 16 bp, synthetic construct
  source 1..16: mol_type = other DNA
ggtttgcacagtaagc

SEQ ID NO: 6 - DNA, 16 bp, synthetic construct
  source 1..16: mol_type = other DNA
ggatggtttgcacagt

SEQ ID NO: 7 - DNA, 16 bp, synthetic construct
  source 1..16: mol_type = other DNA
tacagttttcttggtc

SEQ ID NO: 8 - DNA, 16 bp, synthetic construct
  source 1..16: mol_type = other DNA
gaatttaggtatacag

SEQ ID NO: 9 - DNA, 16 bp, synthetic construct
  source 1..16: mol_type = other DNA
ccaatatagtattctg

SEQ ID NO: 10 - DNA, 16 bp, synthetic construct
  source 1..16: mol_type = other DNA
agttatgtgtaggatt

SEQ ID NO: 11 - DNA, 16 bp, synthetic construct
  source 1..16: mol_type = other DNA
gttatgtgtaggatta

SEQ ID NO: 12 - DNA, 16 bp, synthetic construct
  source 1..16: mol_type = other DNA
gcaaatttgttgatag

SEQ ID NO: 13 - DNA, 16 bp, synthetic construct
  source 1..16: mol_type = other DNA
gacattttacttatcc

SEQ ID NO: 14 - DNA, 16 bp, synthetic construct
  source 1..16: mol_type = other DNA
catgaatggtttgctg

SEQ ID NO: 15 - DNA, 16 bp, synthetic construct
  source 1..16: mol_type = other DNA
attagttatgtgtagg

SEQ ID NO: 16 - DNA, 16 bp, synthetic construct
  source 1..16: mol_type = other DNA
gttatgtgtaggatta

SEQ ID NO: 17 - protein, 23 aa, synthetic construct
DDDATPPPVIAPRPEHTKSVYTR